SEQUENCE LISTING (22 sequences)

SEQ ID NO: 1 - DNA, 1065 bp, Homo sapiens
atgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttga

SEQ ID NO: 2 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 3 - DNA, 32 bp, Artificial sequence
  Synthetic primer
gcgaagcttccaaggagacgggcgctccaggg

SEQ ID NO: 4 - DNA, 33 bp, Artificial sequence
  Synthetic primer
gcgtctagatcatcagagggcgctctggtcttt

SEQ ID NO: 5 - DNA, 33 bp, Artificial sequence
  Synthetic primer
gcggcggccgcccttagtgcgcggaaccagcca

SEQ ID NO: 6 - DNA, 34 bp, Artificial sequence
  Synthetic primer
gcgtctagatcatcagccttgaaatgtacatgtt

SEQ ID NO: 7 - DNA, 31 bp, Artificial sequence
  Synthetic primer
gcgaagcttgcacaacatgagtgtgacgtgg

SEQ ID NO: 8 - DNA, 33 bp, Artificial sequence
  Synthetic primer
gcgtctagatcatcagcagatctgctcctcctt

SEQ ID NO: 9 - DNA, 40 bp, Artificial sequence
  Synthetic primer
gcatcccatcggtccacttcctttgcggcaactttctatg

SEQ ID NO: 10 - DNA, 40 bp, Artificial sequence
  Synthetic primer
catagaaagttgccgcaaaggaagtggaccgatgggatgc

SEQ ID NO: 11 - DNA, 36 bp, Artificial sequence
  Synthetic primer
ctcgctcagcatcccattcctccacttcctttgcgg

SEQ ID NO: 12 - DNA, 36 bp, Artificial sequence
  Synthetic primer
ccgcaaaggaagtggaggaatgggatgctgagcgag

SEQ ID NO: 13 - DNA, 40 bp, Artificial sequence
  Synthetic primer
ccatactcaattatcagcagctccatccagatccctgaag

SEQ ID NO: 14 - DNA, 40 bp, Artificial sequence
  Synthetic primer
cttcagggatctggatggagctgctgataattgagtatgg

SEQ ID NO: 15 - DNA, 55 bp, Artificial sequence
  Synthetic primer
gacgatggcatcgagaagaattcaagagattcttctcgatgccatcgtctttttt

SEQ ID NO: 16 - DNA, 59 bp, Artificial sequence
  Synthetic primer
aattaaaaaagacgatggcatcgagaagaatctcttgaattcttctcgatgccatcgtc

SEQ ID NO: 17 - DNA, 51 bp, Artificial sequence
  Synthetic primer
cacacagatgaatgacaattcaagagattgtcattcatctgtgtgtttttt

SEQ ID NO: 18 - DNA, 55 bp, Artificial sequence
  Synthetic primer
aattaaaaaacacacagatgaatgacaatctcttgaattgtcattcatctgtgtg

SEQ ID NO: 19 - DNA, 20 bp, Artificial sequence
  Synthetic primer
gacgatggcatcgagaagaa

SEQ ID NO: 20 - DNA, 19 bp, Artificial sequence
  Synthetic primer
acacacagatgaatgacaa

SEQ ID NO: 21 - DNA, 4180 bp, Homo sapiens
gcggggaagcagcagcggccaggatgaatcccaggtgctctggagctggatggtgaaggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccag

SEQ ID NO: 22 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

(19) United States
(12) Patent Application Publication
(10) Pub. No.: US 2007/0224194 A1
(43) Pub. Date: Sep. 27, 2007

(54) Methods for Inhibiting Angiogenesis and/or Lymphangiogenesis

(75) Inventors: Bradley McColl, London (GB); Steven Stacker, Melbourne (AU); Marc G. Achen, Melbourne (AU); Karri Paavonen, Parkville (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LUDWIG INSTITUTE FOR CANCER RESEARCH, New York, NY (US)

(21) Appl. No.: 11/566,195

(22) Filed: Dec. 1, 2006

Related U.S. Application Data
(63) Continuation-in-part of application No. 11/133,415, filed on May 20, 2005.
(60) Provisional application No. 60/572,469, filed on May 20, 2004.

(51) Int. Cl. A61K 31/7052; A61K 39/395

(52) U.S. Cl. 424/139.1; 514/1; 514/44

(57) ABSTRACT

Proprotein convertase inhibitor has been found to block proteolytic processing and activation of VEGF-C and VEGF-D and inhibit angiogenesis and/or lymphangiogenesis. Method and composition are disclosed for inhibiting angiogenesis and/or lymphangiogenesis, and for treating conditions associated with excessive angiogenesis, such as tumors and/or retinopathies, as well as conditions associated with lymphangiogenesis, such as the metastatic spread of malignancies, macular degeneration, inflammatory mediated diseases, rheumatoid arthritis, diabetic retinopathy and psoriasis in a patient. The inventive method and composition utilize proprotein convertase antagonist selected from the group consisting of an anti-proprotein convertase antibody, an antisense nucleic acid molecule against a polynucleotide coding for a proprotein convertase, and an siRNA for inhibiting proprotein convertase expression, as well as proprotein convertase inhibitors.

### CROSS REFERENCE TO RELATED APPLICATIONS

This application is a continuation-in-part of U.S. patent application Ser. No. 11/133,415, filed May 2005 which claims the benefit of priority of U.S. Provisional Application No. 60/572,469, filed May 20, 2004. The disclosures of all priority applications are expressly incorporated by reference in their entireties.

### BACKGROUND OF THE INVENTION

Vascular endothelial growth factor-C (VEGF-C) and VEGF-D are secreted glycoproteins that bind and activate VEGF receptor-2 (VEGFR-2) and VEGFR-3 (Achen et al., Proc. Natl. Acad. Sci. USA 95: 548-553, 1998; Joukov et al., EMBO J. 15: 290-298, 1996), cell surface receptor tyrosine kinases expressed predominantly on blood vascular and lymphatic endothelia respectively (for review see Stacker et al., FASEB J. 16: 922-934, 2002). VEGFR-3 signals for lymphangiogenesis (growth of lymphatic vessels) (Veikkola et al., EMBO J. 20: 1223-1231, 2001) whereas VEGFR-2 is thought to signal for angiogenesis (growth of blood vessels). Human VEGF-C and VEGF-D stimulate both angiogenesis and lymphangiogenesis in vivo (Byzova et al., Blood 99: 4434-4442, 2002; Veikkola et al., EMBO J. 20: 1223-1231, 2001; Marconcini et al., Proc. Natl. Acad. Sci. USA 96: 9671-9676, 1999; Rissanen et al., Circ. Res. 92: 1098-1106, 2003; Bhardwaj et al., Human Gene Therapy 14: 1451-1462, 2003; Rutanen et al., Circulation 109: 1029-1035, 2004; Cao et al., Proc. Natl. Acad. Sci. USA 95: 14389-14394, 1998; Jeltsch et al., Science 276: 1423-1425, 1997).

Importantly, the angiogenesis induced by VEGF-C and VEGF-D in tumors can promote solid tumor growth and metastatic spread, and the lymphangiogenesis induced by these growth factors promotes metastatic spread of tumor cells to the lymphatic vessels and lymph nodes (Skobe et al., Nature Med. 7: 192-198, 2001; Stacker et al., Nature Med. 7: 186-191, 2001; Mandriota et al., EMBO J. 20: 672-682, 2001; Karpanen et al., Cancer Res. 61: 1786-1790, 2001; Skobe et al., Am. J. Pathol. 159: 893-903, 2001). Furthermore, clinicopathological data indicate a role for these growth factors in a range of prevalent human cancers. For example, VEGF-D expression was reported to be an independent prognostic factor for both overall and disease-free survival in colorectal cancel (White et al., Cancer Res. 62: 1669-1675, 2002) and levels of VEGF-C mRNA in lung cancer are associated with lymph node metastasis (Niki et al., Clin. Cancer Res. 6: 2431-2439, 2000) and in breast cancer con-elate with lymphatic vessel invasion and shorter disease-free survival (Kinoshita et al., Breast Cancer Res. Treat. 66: 159-164, 2001; for review see Stacker et al., FASEB J. 16: 922-934, 2002 and Stacker et al., Nature Rev. Cancer 2: 573-583, 2002).

VEGF-C and VEGF-D are each secreted from the cell in a full-length form containing an N-terminal propeptide, a C-terminal propeptide and a central VEGF homology domain (VHD) containing the binding sites for VEGFR-2 and VEGFR-3 (Joukov et al., EMBO J. 16: 3898-3911, 1997; Stacker et al., J. Biol. Chem. 274: 32127-32136, 1999). Subsequently, the propeptides are proteolytically cleaved from the VHD to generate a mature form, consisting of dimers of the VHD, that binds VEGFR-2 and VEGFR-3 with high affinity. The affinities of the mature form of VEGF-D for VEGFR-2 and VEGFR-3 are approximately 290-fold and 40-fold greater, respectively, than those of the unprocessed form (Stacker et al., J. Biol. Chem. 274: 32127-32136, 1999) and similar increases in receptor affinity due to processing were observed for VEGF-C (Joukov et al., EMBO J. 16: 3898-3911, 1997). Thus, the proteolytic processing of VEGF-C and VEGF-D is a mechanism for activating these growth factors.

The proprotein convertases are a family of proteases which process precursor proteins by cleaving after the consensus sequence Arg-Xaa-(Lys/Arg)-Arg (for review see Nakayama, Biochem J. 327: 625-635, 1997). This consensus sequence is similar to the sites at which VEGF-C and VEGF-D are cleaved. Furin, the first member of the proprotein convertases to be identified, is potently inhibited by the substrate analogue Decanoyl-Arg-Val-Lys-Arg-chloromethylketone (Dec-RVKR-CMK) (Steineke-Grober et al., EMBO J. 11: 2407-2414, 1992; Sugrue et al., J. Gen. Virol. 82: 1375-1386, 2001; Hallenberger et al., Nature 360: 358-361 1992).

### SUMMARY OF THE INVENTION

It has now been found that treatment with a proprotein convertase inhibitor can block proteolytic processing and activation of VEGF-C and VEGF-D and inhibit angiogenesis and/or lymphangiogenesis.

By reason of their inhibition of angiogenesis and/or lymphangiogenesis, proprotein convertase inhibitors are useful to treat conditions associated with excessive angiogenesis, such as tumors and/or retinopathies, as well as conditions associated with lymphangiogenesis, such as the metastatic spread of malignancies, macular degeneration, inflammatory mediated diseases, rheumatoid arthritis, diabetic retinopathy and psoriasis in a patient.

In one embodiment, this invention provides a method of inhibiting angiogenesis or lymphangiogenesis comprising administering to an organism in need thereof an effective angiogenesis or lymphangiogenesis inhibiting amount of a proprotein convertase antagonist selected from the group consisting of an anti-proprotein convertase antibody, an antisense nucleic acid molecule against a polynucleotide coding for a proprotein convertase, and an siRNA for inhibiting proprotein convertase expression.

Another embodiment of the invention includes a method of inhibiting angiogenesis or lymphangiogenesis comprising administering to an organism in need of inhibition of angiogenesis or lymphangiogenesis a composition that comprises a proprotein convertase inhibitor effective to inhibit angiogenesis or lymphangiogenesis. The organism can be a mammal and is preferably human. In one aspect, the organism has a tumor.

In some aspects, the inhibitor comprises a member selected from the group consisting of antibody substances that bind to a proprotein convertase polypeptide and that inhibit proprotein convertase function; and inhibitory nucleic acids. In one aspect, the proprotein convertase inhibitor is an inhibitor specific for furin.

In one aspect, the inhibitor comprises an antibody substance selected from the group consisting of a monoclonal antibody, antigen binding fragments of proprotein convertase antibodies; and domain antibodies (dAbs). In another aspect, the inhibitor comprises an antibody fragment selected from the group consisting of Fab fragments, F(ab)2 fragments, Fab′ fragments, F(ab′)2 fragments, Fd fragments, Fd′ fragments, Fv fragments, and single chain antibodies. In yet another aspect, the inhibitor comprises a human or humanized antibody substance.

In another aspect, the inhibitor is an inhibitory nucleic acid selected from the group consisting of an antisense oligonucleotide, an inhibitory RNA, a DNA enzyme, a rybozyme and an aptamer. In a related aspect, the inhibitor is a short interfering RNA, a double stranded RNA (dsRNA) or a short hairpin RNA (shRNA). In a particular aspect, the inhibitor is an shRNA comprising at least one sequence selected from the group consisting of SEQ ID NOs: 15, 16, 17 and 18, that targets a portion of the furin cDNA sequence set forth in SEQ ID NO: 19 or SEQ ID NO: 20. Nucleic acid inhibitors also can target nontranslated portions of a gene, including, but not limited to intron sequences.

Another embodiment of the invention includes a method for inhibiting angiogenesis or lymphangiogenesis in an organism, comprising contacting the organism with a double stranded siRNA molecule under conditions suitable to modulate the expression of a proprotein convertase gene in the organism via RNA interference, wherein a first strand of the double stranded siRNA molecule comprises nucleotide sequence having sufficient complementarity to proprotein convertase mRNA, and wherein a second strand of the double stranded siRNA molecule comprises nucleotide sequence having sufficient complementarity to the first strand, for the siRNA molecule to inhibit expression of the proprotein convertase gene via RNA interference.

Also provided are pharmaceutical compositions comprising an effective angiogenesis or lymphangiogenesis inhibiting amount of a proprotein convertase inhibitor, or an antagonist selected from the group consisting of an anti-proprotein convertase antibody, an antisense nucleic acid molecule against a polynucleotide coding for a proprotein convertase, and an siRNA for inhibiting proprotein convertase expression, and a pharmaceutically acceptable excipient.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

The invention will be described in further detail hereinafter with reference to representative embodiments illustrated by the accompanying drawing figures in which:

FIG. 1 is a Western blot analysis of human VEGF-C secreted from cells expressing a tagged, full-length VEGF-C following treatment of the cells with a representative proprotein convertase inhibitor; and

FIG. 2 (and FIG. 7) is a Western blot analysis of human VEGF-D secreted from cells expressing a tagged, full-length VEGF-D following treatment of the cells with a representative proprotein convertase inhibitor.

FIG. 3 shows a schematic map of VEGF-DΔC and VEGF-DΔN (upper panel) and Western blots of the immunoprecipitated VEGF-D proteins from the 293EBNA VEGF-DΔC cells (middle panel) and from the 293EBNA VEGF-DΔN cells (lower panel).

FIG. 4 (and FIG. 8A) shows Western blots comparing the VEGF-D species immunoprecipitated from the conditioned media of (A) 293EBNA and (B) LoVo cells after transient transfection with VEGF-D expression constructs.

FIG. 5 (and FIG. 8B) shows Western blot analysis of conditioned media of LoVo cells co-transfected with VEGF-D expression constructs with PC expression constructs.

FIG. 6 (and FIGS. 9A and B) shows the results of the receptor binding experiments using VEGFR-2-Ig and VEGFR-3-Ig.

FIG. 7 is a Western blot analysis of human VEGF-D secreted from cells expressing a tagged, full-length VEGF-D following treatment of the cells with a representative proprotein convertase inhibitor.

FIG. 8 shows Western blots comparing the VEGF-D species immunoprecipitated from the conditioned media of 293EBNA and LoVo cells after transient transfection with VEGF-D expression constructs. Western blot analysis of conditioned media of LoVo cells co-transfected with VEGF-D expression constructs and PC expression constructs is also provided.

FIG. 9 shows the results of the receptor binding experiments using VEGFR-2-Ig and VEGFR-3-Ig.

FIG. 10 shows a (A) schematic drawing of mutant VEGF-D; (B) proteolytic processing of VEGF-D-Full-N-Flag and VEGF-D_{SSTS.IISS}; (C) binding affinities of various VEGF-D polypeptides to VEGFR-2 and VEGFR-3

### DETAILED DESCRIPTION OF THE INVENTION

A number of proprotein convertase inhibitors are known. Examples of such inhibitors include inhibitory prosegments of proprotein convertases, inhibitory variants of anti-trypsin and peptidyl haloalkylketone inhibitors. Representative inhibitory prosegments of proprotein convertases include the inhibitory prosegments of PC5A (also known as PC6A), PC5B (also known as PC6B), PACE4, PC1 (also known as PC3), PC2, PC4, PC7 and Furin (Thomas, Nature Reviews Mol. Cell Biol. 3 (2002) 753-766; Zhong et al., J. Biol. Chem. 274: 33913-33920, 1999). Furin is a calcium-dependent, membrane-bound serine endoproteinase. It is a member of the “subtilisin-like” proprotein/prohormone convertase (PC) family of enzymes. Furin has a ubiquitous tissue distribution. It cycles between the trans-Golgi network, the cell surface, and the endosomes, directed by defined sequences within furin's cytosolic tail. Furin processes not only intracellular growth factors and serum proteins, but also extracellular matrix proteins and cell surface receptors. Furin has been reported to cleave proproteins at the consensus sequence -Arg-Xaa-Lys/Arg-Arg-↓. The minimum consensus sequence has been reported to be -Arg-Xaa-Xaa-Arg-↓. See H. Angliker, “Synthesis of tight binding inhibitors and their action on the proprotein-processing enzyme furin,” J. Med. Chem., vol. 38. pp. 4014-401 8 (1995). A furin cDNA sequence and deduced amino acid sequence are set forth in SEQ ID NOs: 21 and 22.

A representative inhibitory variant of anti-trypsin is α-1 antitrypsin Portland, an engineered variant of naturally occurring antitrypsin that inhibits multiple proprotein convertases (Jean et al., Proc. Natl Acad. Sci. USA 95 (1998) 7293-7298). Representative peptidyl halomethyl ketone inhibitors include decanoyl-Arg-Val-Lys-Arg-chloromethylketone (Dec-RVKR-CMK), decanoyl-Phe-Ala-Lys-Arg-chloromethylketone (Dec-FAKR-CMK), decanoyl-Arg-Glu-Ile-Arg-chloromethylketone (Dec-REIR-CMK), and decanoyl-Arg-Glu-Lys-Arg-chloromethylketone (Dec-REKR-CMK) (Stieneke-Grober, A. et al., EMBO J. 11 (1992) 2407-2414; Jean et al., Proc. Natl Acad. Sci. USA 95 (1998) 7293-7298; Garten W. et al., Virology 72 (1989) 25-31). For purposes of illustration, the invention will be described hereinafter by reference to exemplary tests using the representative substrate analogue Dec-RVKR-CMK without limiting the invention thereto.

These inhibitors of proprotein convertases, such as Dec-RVKR-CMK or the inhibitory prosegments of PCs, can be used to block the activation of VEGF-C and VEGF-D and thereby inhibit angiogenesis, lymphangiogenesis and other biological effects induced by partially processed or fully processed VEGF-C or VEGF-D. A particularly relevant clinical setting for use of the invention is in the treatment of cancer in which angiogenesis and lymphangiogenesis induced by these growth factors can promote solid tumor growth and/or metastatic spread. However, the invention is also useful in other situations in which angiogenesis and/or lymphangiogenesis underlies the pathology, e.g. macular degeneration in the eye. Other applications include treatment of inflammatory mediated diseases, rheumatoid arthritis, diabetic retinopathy and psoriasis.

Because only the fully processed forms of VEGF-C and VEGF-D bind to VEGF Receptor 2 (VEGFR-2), as well as exhibiting increased binding to VEGF Receptor 3 (VEGFR-3), it is possible by use of proprotein convertase inhibitors to alter the processing of the full length growth factors and thereby modulate the relative rates of VEGFR-2 and VEGFR-3 activation. In this way, one can affect the balance between angiogenesis and lymphangiogenesis through administration of proprotein convertase inhibitors.

The proprotein convertase inhibitors may be administered by known routes of local or systemic administration such as injection to the desired site of action or intravenous administration. The inhibitors may be administered in admixture with known carriers and or adjuvants, as well as with other active agents for the treatment of pathological conditions associated with angiogenesis and/or lymphangiogenesis. Dosage regimens may be determined by persons skilled in the art. For treatment of a human patient, a typical dosage will lie between 0.1 μg and 100 mg per kilogram of body weight.

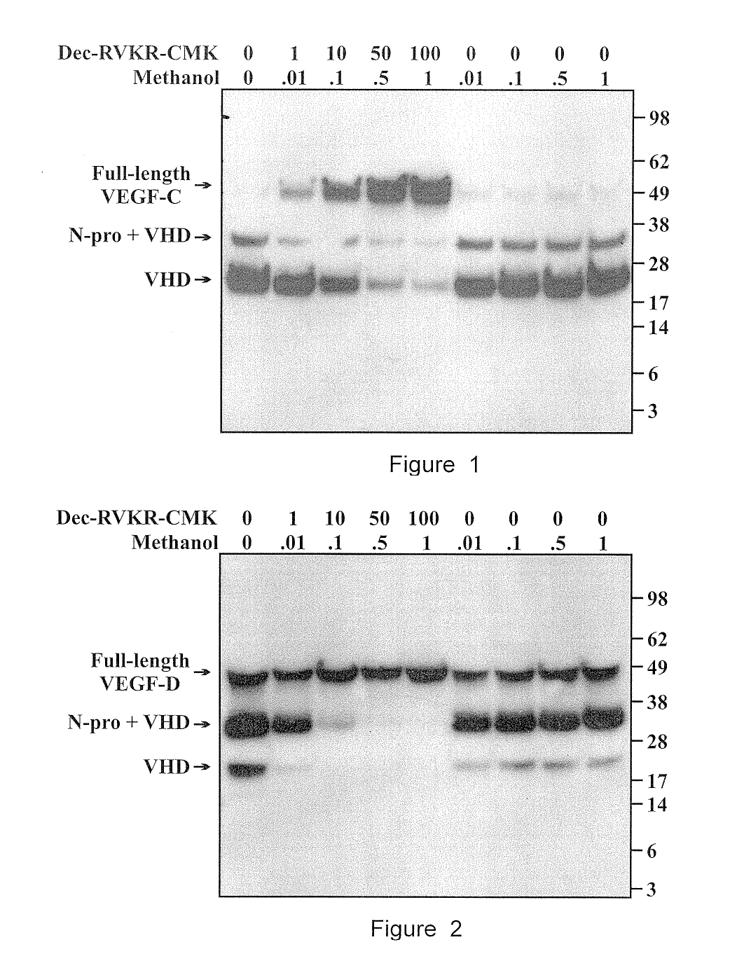

FIGS. 1 and 2 depict the Western blot analysis of human VEGF-C (FIG. 1) and VEGF-D (FIG. 2) secreted from 293EBNA cells expressing VEGF-C-FULL-N-Myc or VEGF-D-FULL-N-FLAG, respectively, following treatment with Dec-RVKR-CMK. The numbers at the top of each figure denote the concentration of Dec-RVKR-CMK (μM), and the concentration of methanol (% v/v). Identities of the various VEGF-C and VEGF-D species are shown to the left. Numbers to the right denote positions of molecular weight markers (kDa).

In addition to the inhibitors described above or otherwise known to those skilled in the art, neutralizing antibodies may also be used to inhibit the biological action (e.g. the function or expression) of proprotein convertase (“the target protein”). In one embodiment of the invention, antisense oligonucleotides are used as antagonizing agents. The antisense oligonucleotides preferably inhibit target expression by inhibiting translation of the target. In a further embodiment, the antagonizing agent is small interfering RNAs (siRNA, also known as RNAi, RNA interference nucleic acids). The siRNA are double-stranded RNA molecules, typically 21 nucleotides in length, that are homologous to a gene or polynucleotide that encodes the proprotein convertase (“the target gene”) and interfere with the target gene's expression. Also provided are methods of inhibiting the target gene expression or target protein function utilizing DNA enzymes; ribozymes and triplex-forming nucleic acid molecules, as will be described in more details below.

An antibody suitable for the present invention may be a polyclonal antibody. Preferably, the antibody is a monoclonal antibody. The antibody may also be isoform-specific. The monoclonal antibody or binding fragment thereof of the invention may be Fab fragments, F(ab)2 fragments, Fab′ fragments, F(ab′)2 fragments, Fd fragments, Fd′ fragments or Fv fragments. Domain antibodies (dAbs) (for review, see Holt et al., 2003, Trends in Biotechnology 21:484-490) are also suitable for the methods of the present invention.

Various methods of producing antibodies with a known antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof may be characterized as those which are capable of specific binding to a target protein or an antigenic fragment thereof, preferably an epitope that is recognized by an antibody when the antibody is administered in vivo. Antibodies can be elicited in an animal host by immunization with a target protein-derived immunogenic component, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be from humans, or from animals other than humans, preferably mammals, such as rat, mouse, guinea pig, rabbit, goat, sheep, and pig. Preferred are mouse monoclonal antibodies and antigen-binding fragments or portions thereof. In addition, chimeric antibodies and hybrid antibodies are embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454. For human therapeutic purposes, humanized, or more preferably, human antibodies are used.

Further, single chain antibodies are also suitable for the present invention (e.g., U.S. Pat. Nos. 5.476,786 and 5,132,405 to Huston; Huston et al., 1988, Proc. Natl. Acad. Sci. USA. 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an animo acid bridge, resulting in a single chain polypeptide. Univalent antibodies are also embraced by the present invention.

Many routes of delivery are known to the skilled artisan for delivery of anti-target antibodies. For example, direct injection may be suitable for delivering the antibody to the site of interest. It is also possible to utilize liposomes with antibodies in their membranes to specifically deliver the liposome to the area where target gene expression or function is to be inhibited. These liposomes can be produced such that they contain, in addition to monoclonal antibody, other therapeutic agents, such as those described above, which would then be released at the tumor site (e.g., Wolff et al., 1984, Biochem. et Biophys. Acta, 802:259).

Inhibitor oligonucleotides of the invention may be, for example: antisense oligonucleotides [Eckstein, Antisense Nucleic Acid Drug Dev., 10: 117-121 (2000); Crooke, Methods Enzymol., 313: 3-45 (2000); Guvakova et al., J Biol. Chem., 270: 2620-2627 (1995); Manoharan, Biochim. Biophys. Acta, 1489: 117-130 (1999); Baker et al., J. Biol. Chem., 272: 11994-12000 (1997); Kurreck, Eur. J. Biochem., 270: 1628-1644 (2003); Sierakowska et al., Proc. Natl. Acad. Sci. USA, 93: 12840-12844 (1996); Marwick, J. Am. Med. Assoc. 280: 871 (1998); Tomita and Morishita, Curr Pharm. Des., 10: 797-803 (2004); Gleave and Monia, Nat. Rev. Cancer 5: 468-479 (2005) and Patil, AAPS J. 7: E61-E77 (2005], triplex oligonucleotides [Francois et al., Nucleic Acids Res., 16: 11431-11440 (1988) and Moser and Dervan, Science, 238: 645-650 (1987)], ribozymes/deoxyribozymes(DNAzymes) [Kruger et al., Tetrahymena. Cell, 31: 147-157 (1982); Uhlenbeck, Nature, 328: 596-600 (1987); Sigurdsson and Eckstein, Trends Biotechnol., 13 286-289 (1995); Kumar et al., Gene Ther, 12: 1486-1493 (2005); Breaker and Joyce, Chem. Biol., 1: 223-229 (1994); Khachigian, Curr. Pharm. Biotechnol., 5: 337-339 (2004); Khachigian, Biochem. Pharmacol., 68: 1023-1025 (2004) and Trulzsch and Wood, J. Neurochem., 88: 257-265 (2004)], small-interfering RNAs/RNAi [Fire et al., Nature, 391: 806-811 (1998); Montgomery et al., Proc. Natl. Acad. Sci. U.S.A., 95: 15502-15507 (1998); Cullen, Nat. Immunol., 3: 597-599 (2002); Hannon, Nature, 418: 244-251 (2002); Bernstein et al., Nature, 409: 363-366 (2001); Nykanen et al., Cell, 107: 309-321 (2001); Gilmore et al., J. Drug Target., 12: 315-340 (2004); Reynolds et al., Nat. Biotechnol., 22: 326-330 (2004); Soutschek et al., Nature, 432173-178 (2004); Ralph et al., Nat. Med., 11: 429-433 (2005); Xia et al., Nat. Med., 10816-820 (2004) and Miller et al., Nucleic Acids Res., 32: 661-668 (2004)], aptamers [Elllington and Szostak, Nature, 346: 818-822 (1990); Doudna et al., Proc. Natl. Acad. Sci. U.S.A., 2355-2359 (1995); Tuerk and Gold, Science, 249-510(1990); White et al., Mol. Ther, 4: 567-573 (2001); Rusconi et al., Nature, 419: 90-94 (2002); Nilajee et al., Mol. Ther., 14: 408-415 (2006); Gragoudas et al., N. Engl. J. Med., 351: 3805-2816 (2004); Vinores, Curr. Opin. Mol. Ther., 5673-679 (2003) and Kourlas and Schiller et al., Clin. Ther, 28 36-44 (2006)] or decoy oligonucleotides [Morishita et al., Proc. Natl. Acad. Sci. U.S.A., 92: 5855-5859 (1995); Alexander et al., J. Am. Med. Assoc., 294: 2446-2454 (2005); Mann and Dzau, J. Clin. Invest., 106: 1071-1075 (2000) and Nimjee et al., Annu. Rev. Med., 56: 555-583 (2005). The foregoing documents are hereby incorporated by reference in their entirety herein, with particular emphasis on those sections of the documents relating to methods of designing, making and using inhibitory oligonucleotides. Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom siRNA molecules. In addition, commercially kits are available to produce custom siRNA molecules, such as SILENCER™ siRNA Construction Kit (Ambion Inc., Austin, Tex.) or psiRNA System (InvivoGen, San Diego, Calif.).

This invention also provides antisense nucleic acid molecules and compositions comprising such antisense molecules. The constitutive expression of antisense RNA in cells has been known to inhibit the gene expression, possibly via the blockage of translation or prevention of splicing. Interference with splicing allows the use of intron sequences which should be less conserved and therefore result in greater specificity, inhibiting expression of a gene product of one species but not its homologue in another species.

The term antisense component corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the target gene so that the antisense RNA can hybridize to the target gene (or mRNA) and inhibit target gene expression regardless of whether the action is at the level of splicing, transcription, or translation. The antisense components of the present invention may be hybridizable to any of several portions of the target cDNA, including the coding sequence, 3′ or 5′ untranslated regions, or other intronic sequences, or to target mRNA.

Antisense RNA is delivered to a cell by transformation or transfection via a vector, including retroviral vectors and plasmids, into which has been placed DNA encoding the antisense RNA with the appropriate regulatory sequences including a promoter to result in expression of the antisense RNA in a host cell. In one embodiment, stable transfection and constitutive expression of vectors containing target cDNA fragments in the antisense orientation are achieved, or such expression may be under the control of tissue or development-specific promoters. Delivery can also be achieved by liposomes.

For in vivo therapy, the currently preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target cDNA, including the coding sequence, 3′ or 5′ untranslated regions, or other intronic sequences, or to target mRNA, are preferred. Sequences for the antisense oligonucleotides to target are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

The preferred target antisense oligonucleotides are those oligonucleotides which are stable, have a high resistance to nucleases, possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In vitro and in vivo assays using antisense oligonucleotides have shown that delivery mediated by cationic liposomes, by retroviral vectors and direct delivery are efficient. Another possible delivery mode is targeting using antibody to cell surface markers for the target cells. Antibody to target or to its receptor may serve this purpose.

Alternatively, nucleic acid sequences which inhibit or interfere with gene expression (e.g., siRNA, shRNA, ribozymes, aptamers) can be used to inhibit or interfere with the activity of RNA or DNA encoding a target protein.

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Compared to siRNA, shRNA offers advantages in silencing longevity and delivery options See, Hannon et al., Nature, 431:371-378, 2004, for review. Vectors that produce shRNAs, which are processed intracellularly into short duplex RNAs having siRNA-like properties have been reported (Brummelkamp et al., Science 296, 550 553, 2000; Paddison et al., Genes Dev. 16, 948-958 (2002). Such vectors provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral or adenoviral vectors, facilitating delivery of shRNAs into a broad range of cell types (Brummelkamp et al., Cancer Cell 2:243-247, 2002; Dirac, et al., J. Biol. Chem. 278:11731-11734, 2003; Michiels et al., Nat. Biotechnol. 20:1154-1157, 2002; Stegmeie et al., Proc. Natl. Acad. Sci. USA 102:13212-13217, 2005; Khvorova et al., Cell, 115:209-216 (2003) in any of the innumerable ways that have been devised for delivery of DNA constructs that allow ectopic mRNA expression. These include standard transient transfection, stable transfection and delivery using viruses ranging from retroviruses to adenoviruses. Expression can also be driven by either constitutive or inducible promoter systems (Paddison et al., Methods Mol. Biol. 265:85-100, 2004). Delivery of nucleic acid inhibitors by replicating or replication-deficient vectors is contemplated as an aspect of the invention.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48: Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411 :428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target Gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:8663-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. Pat. Nos. 6,278,039, 5,723,750 and 5,244,805, which are incorporated herein by reference.

A hairpin can be organized in either a left-handed hairpin (i.e., 5′-antisense-loop-sense-3′) or a right-handed hairpin (i.e., 5′-sense-loop-antisense-3′). Furthermore, the siRNA of the first embodiment may also contain overhangs at either the 5′ or 3′ end of either the sense strand or the antisense strand, depending upon the organization of the hairpin. Preferably, if there are any overhangs, they are on the 3′ end of the hairpin and comprise between 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Preferably, there is only one phosphate group on the 5′ end of the region that will form the antisense strand following Dicer processing. In one aspect, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). The 5′-deoxy chemistry is known to persons skilled in the art, and it is for example described in PCT/US04/10343, which published as WO 2004/090101 A2 on Oct. 21, 2004 and is incorporated by reference herein. In cases where the hairpin is a left-handed hairpin, preferably the 5′ carbon position of the 5′-most nucleotide is phosphorylated.

Hairpins that have stem lengths longer than 26 base pairs can be processed by Dicer such that some of the first region and/or second region may not be part of the resulting siRNA that facilitates mRNA degradation. Accordingly the first region, which may comprise sense nucleotides, and the second region, which may comprise antisense nucleotides, may also contain a stretch of nucleotides that are complementary (or at least substantially complementary to each other), but are or are not the same as or complementary to the target mRNA. While the stem of the shRNA can be composed of complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Optionally, additional modifications can be added to enhance shRNA stability (e.g., including but not limited to those described in the preceding paragraphs), functionality, and/or specificity. Such modified shRNAs can retain the modifications in the post-Dicer processed duplex. For instance, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, this preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of preferred modification patterns are described in detail in U.S. patent application Ser. No. 11/019,831, filed Nov. 22, 2004, with pre-grant publication number 2005/0223427, International Patent application Serial No. PCT/US04/10343, which published as WO 2004/090105 A2 on Oct. 21, 2004, and International Patent application Serial No. PCT/US05/03365 filed on Feb. 4, 2005, the disclosures of which are incorporated by reference herein.

In another non-limiting example of modifications that can be applied to right handed hairpins, 2′-O-methyl modifications (or other 2′ modifications, including but not limited to other 2′-O-alkyl modifications) can be added to nucleotides at position 2, positions 1 and 2, or positions 1, 2, or 3 of the 5′ sense terminus of the hairpin, as well as to the first two (or just the second) nucleotide(s) of the region of the duplex that in the post-Dicer processed molecule represents the 5′ terminus of the antisense strand. The positions of internal chemical modifications can be determined in part by the length of the 3′ overhang. The general rules that define the position of the Dicer cut site, and thus the position of the modifications, are outlined in Vermeulen et al., RNA 11(5), 2005, which is incorporated by reference. Thus, in this example the antisense modifications may, for example, be located on the nucleotides that are complementary to sense nucleotides 19 and 20, 20 and 21, or21 and 22.

Similarly, in cases in which the hairpin is a left-handed hairpin, 2′-O-alkyl modifications can be added to key positions within the molecule such that following Dicer digestion, the 2′-O-methyl groups are associated with: (1) the first and second nucleotides of the 5′ terminus of the sense strand; (2) the first and second nucleotides of the 5′ terminus of the sense strand, plus the first, and optionally second, nucleotides of the antisense strand; or (3) the first and second nucleotides of the 5′ terminus of the sense strand plus the second nucleotide of the antisense sense strand. Addition of chemical modifications in these nucleotide positions can greatly reduce the number of off-targeted genes produced by the sense and/or the sense and antisense strands and/or enhance functionality.

Examples of modifications that can be added to right-handed hairpins to enhance hairpin specificity and functionality can include 5′ deoxy and 5′ blocking modifications. Previous studies have shown that for a strand to participate in RISC mediated RNAi, the 5′ carbon of the 5′ terminal nucleotide must be phosphorylated. As the sense strand of post-Dicer processed shRNA can potentially enter RISC and compete with the antisense (e.g., targeting) strand, modifications that prevent sense strand phosphorylation are valuable in minimizing off-target signatures. Thus, desirable chemical modifications that prevent phosphorylation of the 5′ carbon of the 5′-most nucleotide of right-handed shRNA of the invention can include, but are not limited to, modifications that: (1) add a blocking group (e.g., a 5′-O-alkyl) to the 5′ carbon; or (2) remove the 5′-hydroxyl group (e.g., 5′-deoxy nucleotides). Methods for generating 5′deoxy modified molecules are disclosed in International Patent Application Serial No. PCT/US05/03365, filed Feb. 4, 2005, and published as WO/2005/078094, the disclosure of which is incorporated by reference herein.

In addition to modifications that enhance specificity, modifications that enhance stability can also be added to the invention. In one embodiment, modifications comprising 2′-O-alkyl groups (or other 2′ modifications) can be added to one or more, and preferably all, pyrimidines (e.g., C and/or U nucleotides) of the sense strand. In another embodiment, 2′ F modifications (or other halogen modifications) can be added to one or more, and preferably all pyrimidines (e.g., C and/or U nucleotides) of the antisense strand. In yet a further embodiment, modifications comprising 2′-O-alkyl groups (or other 2′ modifications) can be added to one or more, preferably all, pyrimidines (e.g., C and/or U nucleotides) of the sense strand, plus 2′ F modifications (or other halogen modifications) can be added to one or more, preferably all pyrimidines (e.g., C and/or U nucleotides) of the antisense strand. Modifications such as 2′ F or 2′-O-alkyl of some or all of the Cs and Us of the antisense and/or sense strand/region, respectively, or the loop structure, can greatly enhance the stability of the shRNA molecules without appreciably altering target specific silencing. It should be noted that while these modifications enhance stability, it may be desirable to avoid the addition of these modification patterns to key positions in the hairpin in order to avoid disruption of RNAi (e.g., in and around the Dicer cleavage site).

shRNA may comprise sequences that were selected at random, or according to any rational design selection procedure. For example, the rational design algorithms are described in U.S. patent application Ser. No. 10/714,333, filed on Nov. 14, 2003, entitled “Functional and Hyperfunctional siRNA”; in International Patent Application Serial Number PCT/US2003/036787, which published on Jun. 3, 2004 as WO 2004/045543 A2, entitled “Functional and Hyperfunctional siRNA”; and in U.S. patent application Ser. No. 10/940,892, filed on Sep. 14, 2004, entitled “Methods and Compositions for Selecting siRNA of Improved Functionality,” having pre-grant publication number 2005/0255487, the disclosure of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, 1988, Nature 334:585-591; and PCT Application. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., 1995, Proc. Natl. Acad. Sci. USA, 92:6175-79; de Feyter, and Gaudron, Methods in Molecular Biology, Vol. 74, Chapter 43, “Expressing Ribozymes in Plants”, Edited by Turner. P. C., Humana Press Inc. Totowa, N.J.). In particular, RNA polymerase III-mediated expression of tRNA fusion ribozymes are well known in the art (see Kawasaki et al., 1998, Nature 393:284-9; Kuwabara et al., 1998, Nature Biotechnol. 16:961-5; and Kuwabara et al., 1998, Mol. Cell 2:617-27; Koseki et al., 1999, J. Virol 73:1868-77; Kuwabara et al., 1999, Proc. Natl. Acad. Sci. USA, 96:1886-91; Tanabe et al., 2000, Nature 406:473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

The ribozymes of the present invention also include RNA endoribonucleases (“Cech-type ribozymes”) such as the one which occurs naturally in Tetrahymena thermophila (known as the IVS, or L-19 IVS RNA) and which has been extensively described in Zaug, et al., 1984, Science, 224:574-578; Zaug, et al., 1986, Science 231:470-475; Zaug, et al., 1986, Nature 324:429-433; published International patent application No. WO88/04300; and Been, et al., 1986, Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or siRNA are substantially the same sequence of at least 5 and preferably 6, 7, 8, 9, 10, 11, 12, 13, 14, 15, 16, 17, 18, 19 or 20 or more contiguous nucleotides of a target nucleic acid.

In a long target RNA chain, significant numbers of target sites are not accessible to the ribozyme because they are hidden within secondary or tertiary structures (Birikh et al., 1997, Eur. J. Biochem. 245:1-16). To overcome the problem of target RNA accessibility, computer generated predictions of secondary structure are typically used to identify targets that are most likely to be single-stranded or have an “open” configuration (see Jaeger et al., 1989, Methods Enzymol. 183:281-306). Other approaches utilize a systematic approach to predicting secondary structure which involves assessing a huge number of candidate hybridizing oligonucleotides molecules (see Milner et al., 1997, Nat. Biotechnol. 15: 537-41; and Patzel and Sczakiel, 1998, Nat. Biotechnol. 16:64-8). Additionally, U.S. Pat. No. 6,251,588, the contents of which are herein incorporated by reference, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the siRNA oligonucleotides and ribozymes of the invention.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C., 1991, Anticancer Drug Des., 6:569-84; Helene, C., et al., 1992, Ann. N.Y. Acad. Sci., 660:27-36; and Maher, L. J., 1992, Bioassays 14:807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of target gene. DNA enzymes incorporate some of the mechanistic features of both antisense and ribozyme technologies. DNA enzymes are designed so that they recognize a particular target nucleic acid sequence, much like an antisense oligonucleotide. They are, however, catalytic and specifically cleave the target nucleic acid.

There are currently two basic types of DNA enzymes, both of which were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo are similar methods of delivery RNA ribozyme, as outlined in detail above. Additionally, one of skill in the art will recognize that, like antisense oligonucleotide, DNA enzymes can be optionally modified to improve stability and improve resistance to degradation.

The dosage ranges for the administration of the antagonists of the invention are those large enough to produce the desired effect. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

The antagonists of the invention can be administered parenterally by injection or by gradual perfusion over time. The antagonists can be administered intravenously, intraperitoneally, intramuscularly, subcutaneously, intracavity, or transdermally.

Another embodiment of the present invention relates to pharmaceutical compositions comprising one or more proprotein convertase inhibitor, or an antibody against a proprotein convertase, a suitable antisense nucleic acid molecule against a polynucleotide coding for a proprotein convertase, and an siRNA for inhibiting proprotein convertase expression, together with a physiologically- and/or pharmaceutically-acceptable carrier, excipient, or diluent. Physiologically acceptable carriers, excipients, or stabilizers are known to those skilled in the art (see Remington's Pharmaceutical Sciences, 17th edition, (Ed.) A. Osol, Mack Publishing Company, Easton. Pa., 1985). Acceptable carriers, excipients or stabilizers are nontoxic to recipients at the dosages and concentrations employed, and include butters such as phosphate, citrate, and other organic acids: antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Another embodiment of the present invention pertains to vectors, preferably expression vectors, containing an insert that codes for a nucleic acid or polypeptide inhibitor (including but not limited to antibody polypeptides) of the invention. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. See, McIntyre et al., BMC Biotechnology, 6:1 (2006) for review of design and cloning strategies for constructing shRNA expression vectors,

In general, expression vectors of utility in recombinant DNA techniques are often in the form of plasmids. Accordingly, in one embodiment, an expression vector of the invention is a plasmid. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Thus, in one embodiment, an expression vector of the invention is a viral-based vector. For example, replication defective retroviruses, adenoviruses and adeno-associated viruses can be used. Protocols for producing recombinant retroviruses and for infecting cells in vitro or in vivo with such viruses can be found in Current Protocols in Molecular Biology. Austubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses a regulatable shRNA construct, as described herein, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251 -3260 can be used to express a transactivator fusion protein.

Exemplary vectors of the invention comprise an siRNA or an shRNA-encoding nucleic acid operatively linked to one or more regulatory sequences (e.g., promoter sequences, e.g., Pol II or Pol III promoter sequences). The phrase “operably linked” is intended to mean that the nucleotide sequence of interest (e.g., the shRNA-encoding sequence) is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements, such as transcription termination signals (e.g., polyadenylation signals). Such regulatory sequences are described, for example, in Goeddel; Gene Expression Technology. Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Other elements included in the design of a particular expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The vectors described herein can be introduced into cells or tissues by any one of a variety of known methods within the art. Such methods are described for example in Sambrook et al., Molecular Cloning: A Laboratory Manual, Cold Spring Harbor Laboratory, N.Y. (1992), which is hereby incorporated by reference. See, also, Ausubel et al., Current Protocols in Molecular Biology, John Wiley and Sons, Baltimore, Md. (1989); Hitt et al., “Construction and propagation of human adenovirus vectors,” in Cell Biology: A Laboratory Handbook, Ed. J. E. Celis., Academic Press. 2.sup.nd Edition, Volume 1, pp: 500-512, 1998; Hitt et al., “Techniques for human adenovirus vector construction and characterization,” in Methods in Molecular Genetics, Ed. K. W. Adolph, Academic Press, Orlando, Fla., Volume 7B, pp:12-30, 1995; Hitt, et al., “Construction and propagation of human adenovirus vectors,” in Cell Biology: A Laboratory Handbook,” Ed. J. E. Celis. Academic Press. pp:479-490, 1994, also hereby incorporated by reference. The methods include, for example, stable or transient transfection, lipofection, electroporation and infection with recombinant viral vectors. The term “transfecting” or “transfection” is intended to encompass all conventional techniques for introducing nucleic acid into host cells, including calcium phosphate co-precipitation, DEAE-dextran-mediated transfection, lipofection, electroporation and microinjection. Suitable methods for transfecting host cells can be found in Sambrook et al. (Molecular Cloning: A Laboratory Manual, 2nd Edition, Cold Spring Harbor Laboratory press (1989)), and other laboratory textbooks.

The number of host cells transformed with a nucleic acid of the invention will depend, at least in part, upon the type of recombinant expression vector used and the type of transfection technique used. Nucleic acid can be introduced into a host cell transiently, or more typically, for long term regulation of gene expression, the nucleic acid is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acid of interest. Preferred selectable markers include those which confer resistance to certain drugs, such as G418 and hygromnycin. Selectable markers can be introduced on a separate plasmid from the nucleic acid of interest or, are introduced on the same plasmid. Host cells transfected with a nucleic acid of the invention (e.g., a recombinant expression vector) and a gene for a selectable marker can be identified by selecting for cells using the selectable marker. For example, if the selectable marker encodes a gene conferring neomycin resistance, host cells which have taken up nucleic acid can be selected with G418. Cells that have incorporated the selectable marker gene will survive, while the other cells die.

Nucleic acid encoding a regulatable shRNA can be introduced into eukaryotic cells growing in culture in vitro by conventional transfection techniques (e.g., calcium phosphate precipitation, DEAE-dextran transfection, electroporation etc.). Nucleic acid can also be transferred into cells in vivo, for example by application of a delivery mechanism suitable for introduction of nucleic acid into cells in vivo, such as retroviral vectors (see e.g., Ferry, N et al. (1991) Proc. Natl. Acad. Sci. USA 88:8377-8381; and Kay, M. A. et al. (1992) Human Gene Therapy 3:641-647), adenoviral vectors (see e.g., Rosenfeld, M. A. (1992) Cell 68:143-155; and Herz, J. and Gerard, R. D. (1993) Proc. Natl. Acad. Sci. USA 90:2812-2816), receptor-mediated DNA uptake (see e.g., Wu, G. and Wu, C. H. (1988) J. Biol. Chem. 263:14621; Wilson et al. (1992) J. Biol. Chem. 267:963-967; and U.S. Pat. No. 5,166,320), direct injection of DNA (see e.g., Acsadi et al. (1991) Nature 332: 815-818; and Wolff et al. (1990) Science 247:1465-1468) or particle bombardment (see e.g., Cheng, L. et al. (1993) Proc. Natl. Acad. Sci. USA 90:4455-4459; and Zelenin, A. V. et al. (1993) FEBS Letters 315:29-32). Thus, for gene therapy purposes, cells can be modified in vitro and administered to a subject or, alternatively, cells can be directly modified in vivo.

Another aspect of the invention pertains to host cells into which a host construct of the invention has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells. Other suitable host cells are known to those skilled in the art.

A shRNAscan be used in a functional analysis of the corresponding target RNA. Such a functional analysis is typically carried out in eukaryotic cells, or eukaryotic nonhuman organisms, preferably mammalian cells or organisms. By administering a suitable shRNA molecule, a specific knockout or knockdown phenotype can be obtained in a target cell, e.g. in cell culture or in a target organism.

Thus, another embodiment of the invention includes cells or organisms (e.g., eukaryotic non-human organisms) exhibiting a target gene-specific knockout or knockdown phenotype resulting from a fully or at least partially deficient expression of at least one endogeneous target gene wherein said cell or organism is transfected with or administered, respectively, at least one shRNA, vector comprising DNA encoding said shRNA (or an shRNA precursor) capable of inhibiting the expression of the target gene.

Gene-specific knockout or knockdown phenotypes of cells or non-human organisms, particularly of human cells or non-human mammals may be used in analytic procedures, (including in the functional and/or phenotypical analysis of complex physiological processes such as analysis of gene expression profiles and/or proteomes). Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g. an affinity tag, particularly a multiple affinity tag.

Variants or mutated forms of the target gene differ from the endogeneous target gene in that they encode a gene product which differs from the endogeneous gene product on the amino acid level by substitutions, insertions and/or deletions of single or multiple amino acids. The variants or mutated forms may have the same biological activity as the endogeneous target gene. On the other hand, the variant or mutated target gene may also have a biological activity, which differs from the biological activity of the endogeneous target gene, e.g. a partially deleted activity, a completely deleted activity, an enhanced activity etc. The complementation may be accomplished by compressing the polypeptide encoded by the endogeneous nucleic acid, e.g. a fusion protein comprising the target protein and the affinity tag and the double stranded RNA molecule for knocking out the endogeneous gene in the target cell. This compression may be accomplished by using a suitable expression vector expressing both the polypeptide encoded by the endogenous nucleic acid e.g. the tag-modified target protein and the double stranded RNA molecule or alternatively by using a combination of expression vectors. Proteins and protein complexes which are synthesized de novo in the target cell will contain the exogenous gene product, e.g., the modified fusion protein. In order to avoid suppression of the exogenous gene product by the siRNA molecule, the nucleotide sequence encoding the exogenous nucleic acid may be altered at the DNA level (with or without causing mutations on the amino acid level) in the part of the sequence which is homologous to the siRNA molecule. Alternatively, the endogeneous target gene may be complemented by corresponding nucleotide sequences from other species, e.g. from mouse.

### EXAMPLES

### Example 1

### Inhibition of Proteolytic Processing of VEGF-C and VEGF-D by Dec-RVKR-CMK

293EBNA cells stably transfected with an expression construct encoding VEGF-C-FULL-N-Myc (full-length VEGF-C tagged with Myc at the N-terminus) or VEGF-D-FULL-N-FLAG (full-length VEGF-D tagged with FLAG at the N-terminus) secrete fully and partially processed forms of VEGF-C or VEGF-D into the culture medium (Joukov et al., EMBO J. 16:3898-3911 1997; Stacker et al., J. Biol. Chem. 274: 32127-32136, 1999). These cells were grown overnight in 24-well plates, seeded with 8×10⁴ cells per well. At the time when cultures were initiated in these plates, growth medium was supplemented with Dec-RVKR-CMK (Calbiochem) (dissolved in methanol) to a final concentration of 0, 1, 10, 50 or 100 μM. Solvent control cultures were also established, in which the medium was supplemented with methanol at concentrations equal to those present in the cultures treated with inhibitor. After 18 hours of growth (at 37° C., 10% CO₂ in a humidified environment), conditioned cell culture medium was removed from the cells and cleared by centrifugation (5 minutes, 250×g, 4° C.).

VEGF-C species were immunoprecipitated from conditioned medium by addition of anti-VEGF-C antibody that binds the VHD and C-terminal propeptide (R&D Systems). VEGF-D species were immunoprecipitated by addition of A2 antiserum that binds within the VHD of VEGF-D (Stacker et al., J. Biol. Chem. 274: 32127-32136, 1999). Immune complexes were allowed to form for 2 hours at 4° C. with gentle agitation and precipitated by addition of 10 μl of protein A sepharose beads and incubation for 2 hours at 4° C. with gentle agitation. Protein A sepharose complexes were recovered by centrifugation (5 minutes, 250×g, 4° C.) and washed twice with ice cold 50 mM Tris, 150 mM NaCl, pH 8.0. The precipitated VEGF-C and VEGF-D species were then analysed by Western blot using biotinylated antibodies against the VHD and C terminus of VEGF-C, or the VHD of VEGF-D (R&D Systems) and streptavidin-horse radish peroxidase conjugate (Zymed) (FIGS. 1 and 2).

FIG. 1 shows a Western blot of immunoprecipitated samples from 293 VEGF-C-FULL-N-Myc cells. The bands in the control sample (extreme left lane) have been characterised previously (Joukov et al., EMBO J. 16: 3898-3911 1997), and are as follows: the 50 kDa band is full-length VEGF-C, the 33 kDa band is the VHD bound to the N-terminal propeptide (N-pro) and the 22 kDa band is the mature VEGF-C subunit consisting of the VHD.

FIG. 2 shows a Western blot of immunoprecipitated samples from 293 VEGF-D-FULL-N-FLAG cells. The bands in the control sample (extreme left lane) have been characterised previously (Stacker et al., J. Biol. Chem. 274: 32127-32136, 1999) and are as follows: the 48 kDa band is full-length VEGF-D, the 33 kDa species is the VHD bound to the N-terminal propeptide (N-pro) and the 21 kDa band is the mature VEGF-D subunit consisting of the VHD.

As a result of treatment of 293EBNA cells expressing either VEGF-C-FULL-N-Myc or VEGF-D-FULL-N-FLAG with Dec-RVKR-CMK, the fully and partially processed forms of VEGF-C and VEGF-D are dramatically reduced in abundance in a dose-dependent manner. At the highest concentration of Dec-RVKR-CMK (100 μM), only the full-length form of VEGF-D was detected indicating that proteolytic processing had been totally blocked. Cells treated with methanol did not show altered processing of VEGF-C or VEGF-D.

These results demonstrate that treatment with a proprotein convertase inhibitor can block proteolytic processing and activation of VEGF-C and VEGF-D. Thus, by blocking this activation in vivo with proprotein convertase inhibitors, it is possible to treat conditions associated with VEGF-C and/or VEGF-D activity, for example, conditions associated with angiogenesis and/or lymphangiogenesis.

### Example 2

### An Inhibitor of All PCs Blocks Processing of VEGF-D at Both the N-terminal and C-terminal Sites of Cleavage

VEGF-D is proteolytically processed at the N- and C-termini of the VHD to generate a mature form consisting of dimers of the VHD (Stacker et al, J. Biol. Chem. 274 32127-32136, 1999). The effect of Dec-RVKR-CMK, an inhibitor of all PCs (Jean et al, Proc. Natl. Acad. Sci. USA 95 7293-7298 1998), on the processing of VEGF-D derivatives ( (i) full-length human VEGF-D tagged at the N-terminus with the FLAG epitope (VEGF-D-FULL-N-FLAG; (ii) VEGF-DΔN-FLAG, a derivative of human VEGF-D lacking the N-terminal propeptide that is tagged with the FLAG epitope at the N-terminus of the VHD, and (iii) VEGF-DΔC-FLAG, a derivative of human VEGF-D in which the C-terminal propeptide has been deleted and replaced with the FLAG epitope) by 293EBNA cells was monitored to establish if PCs can carry out both of these cleavage events.

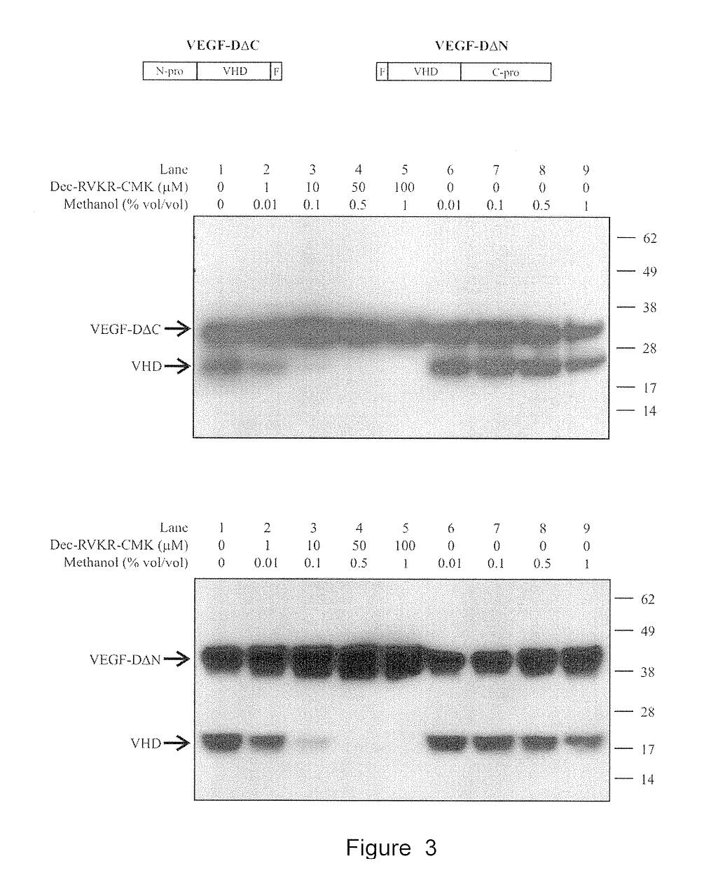

An expression plasmid encoding a truncated derivative of human VEGF-D consisting of the N-terminal propeptide and the VHD tagged at the carboxyl terminus with the FLAG epitope (referred to as VEGF-DΔC) (FIG. 3, top panel) has been previously described (Stacker et al, J. Biol. Chem. 274 32127-32136, 1999). The VEGF-DΔC protein construct facilitates study of cleavage of the N-terminal propeptide from the VHD. The conditioned media of 293EBNA cells stably transfected with the VEGF-DΔC expression plasmid contain a mixture of VEGF-D proteins, including unprocessed VEGF-DΔC, and the proteolytically processed VHD. A second cell line was generated by stable transfection of 293EBNA cells with an expression construct encoding VEGF-DΔN, a truncated derivative of human VEGF-D consisting of the VHD tagged at the amino terminus with the FLAG epitope and C-terminal propeptide (FIG. 3, top panel). This protein construct facilitates study of cleavage of the C-terminal propeptide from the VHD. The conditioned media of 293EBNA cells expressing VEGF-DΔN contain a mixture of VEGF-D proteins, consisting of unprocessed VEGF-DΔN, and the proteolytically processed VHD. The VEGF-DΔN expression plasmid was constructed by replacing the 2-kb EcoRV fragment of the pAPEX-3 construct for full-length VEGF-D with the 2-kb EcoRV fragment of the pAPEX-3 construct for VEGF-DΔNΔC-FLAG (Stacker et al, J. Biol. Chem. 274 32127-32136, 1999).

The 293EBNA cell lines expressing VEGF-D-FULL-FLAG, VEGF-DΔC or VEGF-DΔN were seeded into 24-well plates (8×10⁴ cells/well), in medium supplemented with Dec-RVKR-CMK dissolved in methanol, to a final concentration of 0, 1, 10, 50 or 100 μM. After 18 hours incubation (37° C., 10% CO₂ in an humidified incubator), conditioned cell culture media were removed and clarified by centrifugation (5 minutes, 250×g, 4° C.). VEGF-D species were immunoprecipitated from conditioned media of 293EBNA VEGF-DΔC cells by addition of M2-agarose beads (Sigma-Aldrich), that bind the FLAG epitope attached to the carboxyl-terminus of the VHD, and from conditioned media of 293EBNA VEGF-DΔN cells by addition of A2 antiserum, that binds to epitopes within the VHD (Stacker et al, J. Biol. Chem. 274 32127-32136, 1999), followed by incubation with protein-A sepharose beads. Beads and bound proteins were recovered by centrifugation (5 minutes, 250×g, 4° C.) and washed twice with Tris-buffered saline (TBS; 50 mM Tris-Cl, 150 mM NaCl, pH 8.0). The precipitated VEGF-D species were then analysed by reducing SDS-PAGE and Western blotting using a biotinylated antibody against the VHD of human VEGF-D (R&D Systems), and Streptavidin-horse radish peroxidase conjugate (Zymed).

FIG. 3 shows a schematic map of VEGF-DΔC and VEGF-DΔN (upper panel) and Western blots of the immunoprecipitated VEGF-D proteins from the 293EBNA VEGF-DΔC cells (middle panel) and from the 293EBNA VEGF-DΔN cells (lower panel). In the top panel, “F” denotes the FLAG peptide, “N-pro” the N-terminal propeptide and “C-pro” the C-terminal propeptide. For the middle panel, bands in the control sample (lane 1) consist of unprocessed VEGF-DΔC (˜33 kDa) and the VHD (˜21 kDa) generated by proteolytic removal of the N-terminal propeptide. Following treatment of the 293EBNA VEGF-DΔC cells with Dec-RVKR-CMK, the band representing mature VHD is reduced in abundance in a dose dependent manner (lanes 2-5). At the highest concentration of Dec-RVKR-CMK used (100 μM; lane 5) only unprocessed VEGF-DΔC is detected, indicating that proteolytic processing is completely blocked. For the lower panel, the bands in the control sample (lane 1) consist of unprocessed VEGF-DΔN (˜44 kDa) and the VHD (˜21 kDa) generated by proteolytic removal of the amino-terminal propeptide. Following treatment of the 293EBNA VEGF-DΔN cells with Dec-RVKR-CMK the band representing mature VHD is reduced in abundance in a dose dependent manner (lanes 2-5). At the highest concentration of Dec-RVKR-CMK used (100 μM; lane 5) only unprocessed VEGF-DΔN DΔN is detected, indicating that proteolytic processing is completely blocked. For both the middle and lower panels, cells treated with methanol alone, to control for solvent-specific effects, did not show altered processing of VEGF-D (lanes 6 9). Concentrations of Dec-RVKR-CMK (μM) and methanol (% vol/vol) are show n above these panels, the identities of VEGF-D species to the left and sizes of molecular weight standards (kDa) to the right.

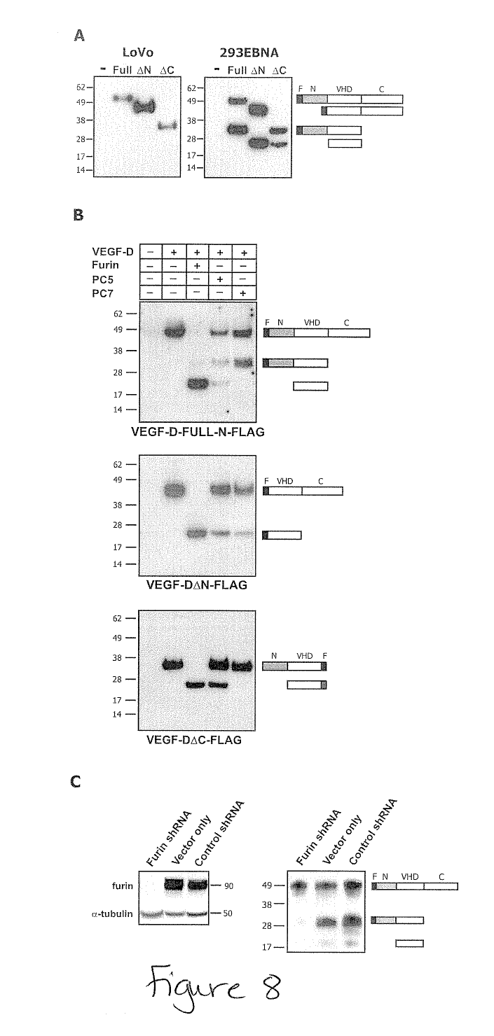

FIG. 7 is a schematic map of VEGF-D-FULL-N-FLAG, VEGF-DΔN and VEGF-DΔC and Western blots of the immunoprecipitated VEGF-D proteins from the 293EBNA VEGF-D-FULL-N-FLAG, cells, VEGF-DΔC cells and from the 293EBNA VEGF-DΔN cells.

Treatment of the 293EBNA VEGF-D-FULL-N-FLAG cells with 100 μM dec-RVKR-CMK (FIG. 7A) resulted in complete inhibition of VEGF-D processing, as demonstrated by the absence of the lower molecular weight VEGF-D species generated by proteolysis, namely the 31 kDa species consisting of the N-terminal propeptide and VHD and of the ˜21 kDa VHD species. At lower concentrations of dec-RVKR-CMK the proteolysis of VFGF-D-FULL-N-FLAG was incompletely inhibited, indicating that the effect of the inhibitor was dose-dependent.

Next, the effect of dec-RVKR-CMK on processing of VEGF-DΔN-FLAG and VEGF-DΔC-FLAG was examined to monitor individually the role of PCs in the cleavage of each propeptide (FIGS. 7B and C). In the absence of the inhibitor, a portion of these proteins is processed to generate the ˜21 kDa mature VHD. However, the inhibitor blocked each of these processing events in a dose-dependent fashion. These findings demonstrate that dec-RVKR-CMK blocks cleavage of both the N- and C-terminal propeptides from VEGF-D, and suggest that members of the PC family play a role in promoting these proteolytic events.

Finally, the effect of dec-RVKR-CMK on VEGF-D processing by Balbc/3T3 cells that naturally produce this growth factor (i.e., in cells that had not been transfected so as to express VEGF-D) was examined. Incubation of these cells with 100 μM dec-RVKR-CMK almost completely blocked VEGF-D propeptide processing (FIG. 7D), indicating the involvement of the PC family in processing of VEGF-D by Balbc/3T3 cells.

These results show that an inhibitor of all the PCs blocks the cleavage of both the N- and C-terminal propeptides from the VHD of VEGF-D, indicating that members of this protease family may be important for activation of this growth factor.

### Example 3

### VEGF-D is not Processed in LoVo Cells Lacking Active Furin

The LoVo cell line is a human colon carcinoma line that lacks enzymatically active furin (Takahashi et al, Biochem. Biophys. Res. Commun. 195 1019-1026 1993; Takahashi et al, J. Biol. Chem. 270 26565-26569, 1995), a broadly expressed member of the proprotein convertases (Nakayama, Biochem. J. 327 625-635, 1997). As a result, LoVo cells fail to process numerous proproteins (Nakayama, Biochem. J. 327 625-635, 1997). Therefore, LoVo cells were analyzed for the capacity to process VEGF-D to establish if this cell line could be used as a processing-deficient background in which to monitor the effect of individual PCs on the proteolytic activation of VEGF-D.

LoVo and 293EBNA cells were transiently transfected with expression constructs encoding full-length VEGF-D tagged at the amino terminus with the FLAG epitope (pVDAPEX FULL-N-FLAG), VEGF-DΔN tagged at the amino terminus with the FLAG epitope (pVDAPEXΔN), or VEGF-DΔC tagged at the carboxyl terminus with the FLAG epitope (pVDAPEXΔC) (Stacker et al, J. Biol. Chem. 274 32127-32136, 1999). After 48 hours incubation (37° C., 10% CO₂ in an humidified incubator), conditioned media were immunoprecipitated by addition of M2-agarose beads (Sigma-Aldrich) that bind the FLAG epitope. Immune complexes were allowed to form for two hours at 4° C. with gentle agitation. Beads and bound proteins were recovered by centrifugation (5 minutes, 250×g, 4° C.) and washed twice with Tris-buffered saline (TBS; 50 mM Tris-Cl, 150 mM NaCl, pH 8.0). The precipitated VEGF-D species were then analysed by reducing SDS-PAGE and Western blotting using an M2-horse radish peroxidase conjugate (Sigma-Aldrich).

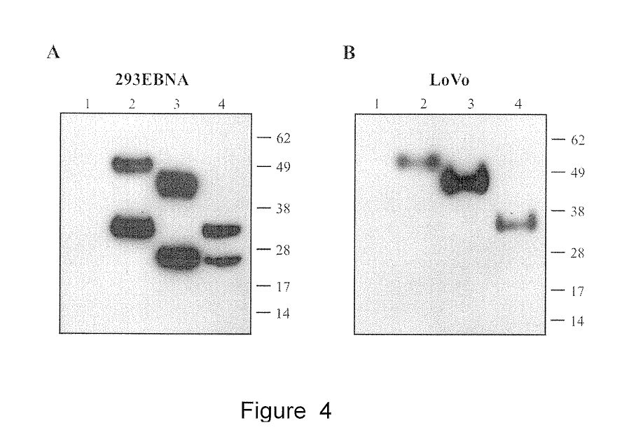

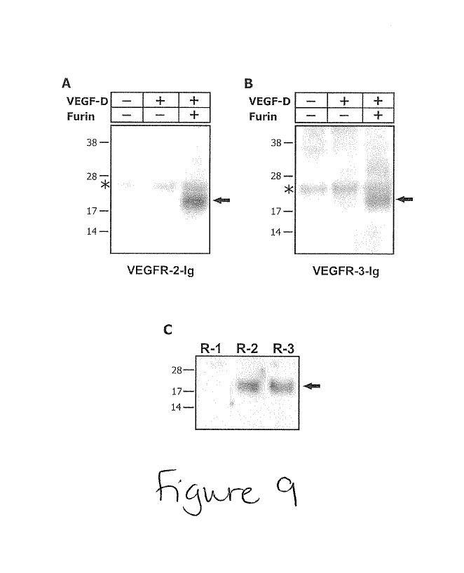

FIGS. 4A and B (and FIG. 8A) show Western blots comparing the VEGF-D species immunoprecipitated from the conditioned media of (A) 293EBNA and (B) LoVo cells after transient transfection with VEGF-D expression constructs. Expression constructs used to transfect cells are as follows: Lane 1 pAPEX3; lane 2 pVDAPEX FULL-N-FLAG; lane 3 pVDAPEXΔN; lane 4 pVDAPEXΔC. Proteolytic processing in 293EBNA cells leads to detection of unprocessed (˜48 kDa band in lane 2, ˜44 kDa band in lane 3 and ˜33 kDa band in lane 4) and processed VEGF-D derivatives (˜33 kDa band in lane 2, ˜21 kDa band in lane 3 and ˜21 kDa band in lane 4). In contrast, when expressed in LoVo cells, only the unprocessed VEGF-D derivatives are detected. No processed forms of the proteins were detected in the conditioned media of the LoVo cells even after prolonged exposure of the blots. Sizes of molecular weight standards (kDa) are shown to the right.

These findings demonstrate that LoVo cells are incapable of processing VEGF-D and that this cell line will be an appropriate background for co-expression studies aimed at identifying the PCs capable of processing VEGF-D.

### Example 4

### Processing of VEGF-D by Individual PCs

In order to establish which members of the PC family of proteases are capable of activating VEGF-D, LoVo cells were co-transfected with expression constructs for VEGF-D derivatives and for various PCs, and the transfected cells analysed for the capacity to process VEGF-D.

Genbank Accession Nos. for furin, PC5 and PC7 mRNA are NM_{—}002569.2, NM_{—}006200, and NM_{—}004716, respectively. Proprotein convertase expression plasmids were constructed by polymerase chain reaction (PCR) amplification of the open reading frames of furin, PC5 and PC7. Cloned cDNAs encoding these proteases were supplied by the American Type Culture Collection and used as template DNA for the amplification reactions. Oligonucleotides used for the amplification of the open reading frames contained restriction enzyme sites to facilitate cloning of PCR products. The coding regions for human furin, PC5 and PC7 were amplified by PCR with Pfx DNA polymerase (Invitrogen) The oligonucleotides used were, for furin:

for furin:

(SEQ ID NO:3)

5′-GCG AAG CTT CCA AGG AGA CGG GCG CTC CAG GG
and

(SEQ ID NO:4)

5′-GCG TCT AGA TCA TCA GAG GGC GCT CTG GTC TTT;
for PC5:

(SEQ ID NO:5)

5′-GCG GCG GCC GCC CTT AGT GCG CGG AAC CAG CCA
and

(SEQ ID NO:6)

5′-GCG TCT AGA TCA TCA GCC TTG AAA TGT ACA TGT T;
and
for PC7:

(SEQ ID NO:7)

GCG AAG CTT GCA CAA CAT GAG TGT GAC GTG G
and

(SEQ ID NO:8)

5′-GCG TCT AGA TCA TCA GCA GAT CTG CTC CTC CTT.

The PCRs involved an initial incubation for 5 min. at 94° C., followed by 15 cycles of 94° C. for 30 seconds (denaturation) followed by 52° C. for 30 s (annealing) and 68° C. for 3 min. (extension). PCR products were cloned into pcDNA3 (Invitrogen, Carlsbad USA) by digestion of PCR products with the appropriate restriction enzymes and ligation to similarly digested pcDNA3 with T4 DNA ligase. The amplified open reading frames of furin and PC7 were ligated into pcDNA3 after digestion with HindIII and XbaI. The amplified open reading frame of PC5 was ligated into pcDNA3 after digestion with NotI and XbaI. Recombinant plasmids shown to contain the desired DNA fragment by restriction mapping were sequenced on both strands to exclude the presence of mutations. The proprotein convertase expression constructs were designated pcDNA3:furin, pcDNA3:PC5 and pcDNA3:PC7.

The proprotein convertase expression constructs were used to co-transfect LoVo cells in combination with pVDAPEX FULL-N-FLAG, pVDAPEXΔN or pVDAPEXΔC. After 48 hours incubation (37° C., 10% CO₂ in an humidified incubator), conditioned media were removed and clarified by centrifugation (5 minutes, 250×g, 4° C.). VEGF-D species from cells transfected with PC expression constructs in combination with pVDAPEX FULL-N-FLAG were immunoprecipitated from conditioned media by addition of A2 antiserum that binds to epitopes within the VHD (Stacker et al, J. Biol. Chem. 274 32127-32136, 1999). Immune complexes were allowed to form for two hours at 4° C. with gentle agitation, and precipitated by addition of 10 μl of protein-A sepharose beads, and incubation for a further two hours as before. Conditioned media from LoVo cells transfected with PC expression constructs in combination with pVDAPEXΔN or pVDAPEXΔC were immunoprecipitated with M2-agarose beads (Sigma-Aldrich). Precipitated proteins were recovered by centrifugation (5 minutes, 250Δg, 4° C.), washed twice with Tris-buffered saline (TBS; 50 mM Tris-Cl, 150 mM NaCl, pH 8.0) and analysed by reducing SDS-PAGE and Western blotting using a biotinylated antibody against the VHD of human VEGF-D (R&D Systems) followed by Streptavidin-horse radish peroxidase conjugate (Zymed) in the case of material from cells transfected with pVDAPEX FULL-N-FLAG, or using an M2-horse radish peroxidase conjugate (Sigma-Aldrich) in the case of material from cells transfected with pVDAPEXΔN or pVDAPEXΔC.

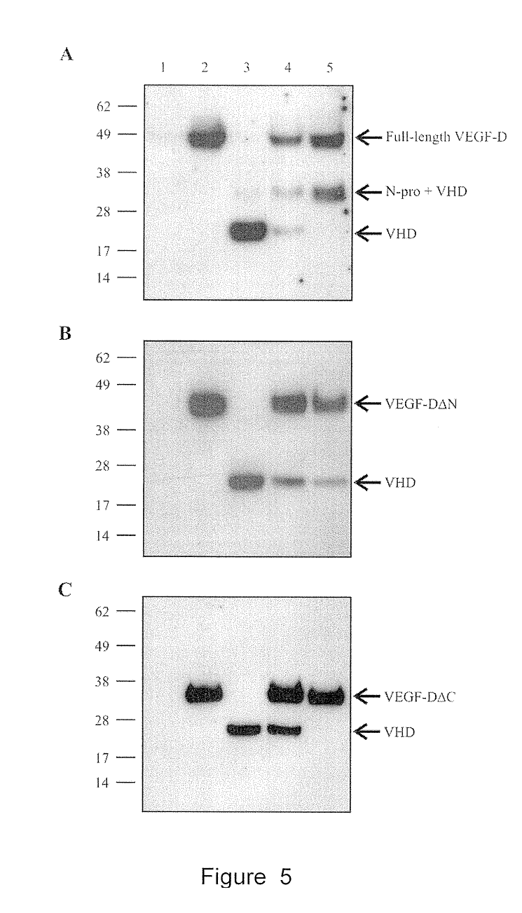

FIG. 5 (and FIG. 8B) shows Western blot analysis of conditioned media of LoVo cells co-transfected with VEGF-D expression constructs with PC expression constructs. Sizes of molecular weight standards (kDa) are shown to the left of the figures and the identities of the bands to the right. FIG. 5A shows the results of co-transfection of LoVo cells with pVDAPEX FULL-N-FLAG in combination with expression constructs encoding human furin, PC5 or PC7. The DNA combinations used to transfect LoVo cells were as follows: Lane 1, pAPEX3+pcDNA3; lane 2, pVDAPEX FULL-N-FLAG+pcDNA3; lane 3, pVDAPEX FULL-N-FLAG+pcDNA3:furin; lane 4, pVDAPEX FULL-N-FLAG+pcDNA3:PC5; lane 5, pVDAPEX FULL-N-FLAG+pcDNA3:PC7. No processing of VEGF-D-FULL-N-FLAG was observed in the absence of PC expression constructs (lane 2). The introduction of pcDNA3:furin resulted in almost complete conversion of the ˜48 kDa VEGF-D FULL-N-FLAG protein to the ˜21 kDa mature VHD species (lane 3). Co-transfection with pcDNA3:PC5 resulted in three fragments being detected in the conditioned medium, unprocessed VEGF-D FULL-N-FLAG (˜48 kDa), a partially processed form consisting of the amino-terminal propeptide and the VHD (˜33 kDa), and fully processed mature VHD (˜21 kDa) (lane 4). These findings indicate that furin and PC5 can cleave both propeptides for the VHD of VEGF-D. Co-transfection of pVDAPEX FULL-N-FLAG with pcDNA3 :PC7 generated unprocessed protein and the partially processed ˜33 kDa form but not the mature VHD (lane 5) (the mature VHD was not detected even after prolonged exposure of the blot) suggesting that PC7 can remove the C-terminal propeptide, but not the N-terminal propeptide from the VHD.

FIG. 5B shows the results of co-transfection of LoVo cells with pVDAPEXΔN and expression constructs encoding furin, PC5 or PC7. DNA combinations used to transfect cells were as follows: Lane 1, pAPEX3+pcDNA3; lane 2, pVDAPEXΔN+pcDNA3; lane 3, pVDAPEXΔN+pcDNA3:furin; lane 4, pVDAPEXΔN+pcDNA3:PC5; lane 5, pVDAPEXΔN+pcDNA3:PC7. LoVo cells did not process VEGF-DΔN in the absence of PC expression constructs, as a single ˜44 kDa species was detected in the conditioned media (lane 2). Co-transfection of LoVo cells with pVDAPEXΔN and pcDNA3:furin resulted in complete conversion of VEGF-DΔN to the 21 kDa mature VHD species (lane 3). The presence of pcDNA3:PC5 or pcDNA3:PC7 resulted in a portion of VEGF-DΔN being processed to the mature VHD (lanes 4 and 5). These results demonstrate that furin, PC5 and PC7 are all capable of promoting the proteolytic cleavage of the C-terminal propeptide from the VHD of VEGF-DΔN.

FIG. 5C shows co-transfection of LoVo cells with pVDAPEXΔC and expression constructs encoding furin, PC5 or PC7. The DNA combinations used to transfect LoVo cells were as follows: Lane 1, pAPEX3+pcDNA3; lane 2, pVDAPEXΔC+pcDNA3; lane 3, pVDAPEXΔC+pcDNA3:furin; lane 4, pVDAPEXΔC+pcDNA3:PC5; lane 5, pVDAPEXΔC+pcDNA3:PC7. In the absence of the PC expression constructs, a single ˜33 kDa protein band was detected, corresponding to intact VEGF-DΔC (lane 2). Co-transfection with pVDAPEXΔC and pcDNA3:furin resulted in a single ˜21 kDa species representing the mature VHD (lane 3). Processing to generate the mature VHD was also detected in the conditioned medium of cells co-transfected with pVDAPEXΔC and the pcDNA3 :PC5 (lane 4). However, co-transfection of pVDAPEXΔC and the pcDNA3:,PC7 expression construct did not result in processing of VEGF-DΔC (lane 5).

These results demonstrate that furin can promote cleavage of both the N-terminal and C-terminal propeptides from the VHD of VEGF-D, and can completely convert VEGF-DΔN to the mature VHD species, whereas PC5 and P7 were much less effective. The more restricted activity of PC7 is in agreement with the observation that a pair of adjacent basic residues, immediately preceding the scissile peptide bond, is essential for PC7 activity (van de Loo et al., J. Biol. Chem., 272:27116-27123, 1997). Such a pair of basic residues is not present in either the “Major” or “Minor” N-terminal propeptide cleavage sites of VEGF-D.

### Example 5

### The mature form of VEGF-D generated by furin binds VEGFR-2 and VEGFR-3

The mature form of VEGF-D generated by 293EBNA cells is an activating ligand for the receptor tyrosine kinases VEGFR-2 and VEGFR-3 (Achen et al, Proc. Natl. Acad. Sci. USA 95 548-553, 1998). Therefore, receptor binding studies were carried out to establish if the mature form of VEGF-D generated by furin in transfected LoVo cells is a ligand for these receptors.

Binding to receptor extracellular domains was examined using soluble fusion proteins consisting of the extracellular domains of human VEGFR-1, VEGFR-2 or VEGFR-3 joined to the Fe portions of human IgG (referred to as VEGFR-1-Ig (R&D Systems), VEGFR-2-Ig and VEGFR-3-Ig) (Achen et al, Proc. Natl. Acad. Sci. USA 95 548-553, 1998). Protein-A sepharose beads were incubated overnight with the conditioned media of 293EBNA cells expressing VEGFR-1-Ig, VEGFR-2-Ig or VEGFR-3-Ig to precipitate the soluble receptor constructs. The soluble receptors bound to protein-A sepharose beads were recovered by centrifugation (5 minutes, 250×g, 4° C.) and washed twice with binding buffer (0.5% (w/v) BSA, 0.02% (v/v) Tween 20, 10 μg/ml heparin in phosphate buffered saline). The soluble receptors bound to protein-A sepharose beads were then incubated with the conditioned media of LoVo cells, that had been co-transfected with pVDAPEX FULL-N-FLAG and pcDNA3:furin, for three hours at room temperature, and washed twice with binding buffer. Proteins bound to the VEGFR-2-Ig and VEGFR-3-Ig constructs were analysed by reducing SDS-PAGE and Western blotting with a biotinylated antibody against the VHD of human VEGF-D (R&D Systems), followed by Streptavidin-horse radish peroxidase conjugate (Zymed).

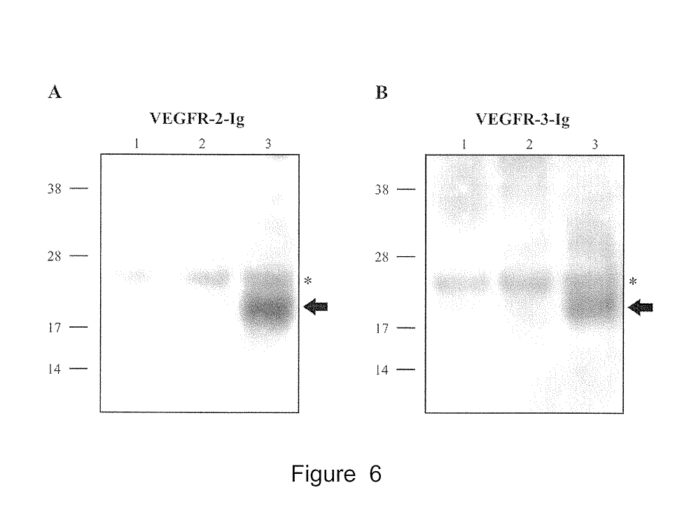

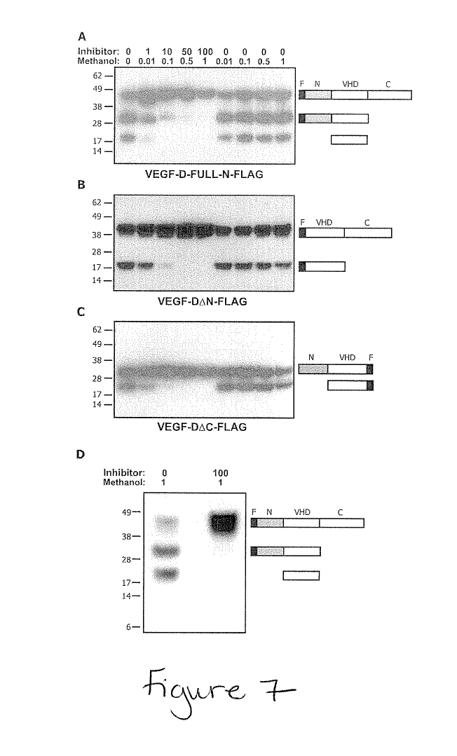

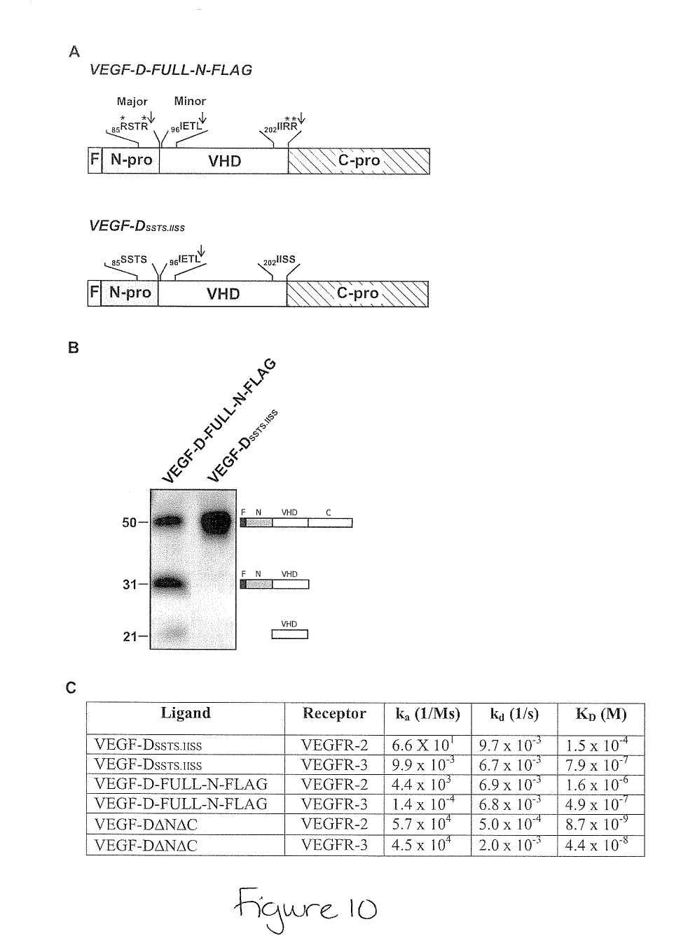

FIG. 6 (and FIGS. 9A and 9B) shows the results of the receptor binding experiments using (A) VEGFR-2-Ig and (B) VEGFR-3-Ig. No mature VEGF-D species were detected in the medium of cells transfected with pAPEX3 and pcDNA3 (lane 1), nor with pVDAPEX FULL-N-FLAG and pcDNA3 (lane 2). Both VEGFR-2-Ig and VEGFR-3-Ig precipitated an ˜21 kDa VEGF-D species from the medium of LoVo cells transfected with pVDAPEX FULL-N-FLAG and pcDNA3:furin (lane 3, indicated by arrows). Sizes of molecular weight standards are shown to the left of each panel and asterisks indicate a non-specific band observed in all samples.

FIG. 9C, VEGFR-1-Ig (R-1), VEGFR-2-Ig (R-2) and VEGFR-3-Ig (R-3) were used to precipitate mature VEGF-D from the conditioned media of LoVo cells cotransfected with expression vectors for VEGF-D-FULL-N-FLAG and furin. Precipitated proteins were analyzed by reducing SDS-PAGE and Western blotting with antibodies that bind to the VHD of VEGF-D. The arrow in FIG. 9 indicates the mature form of VEGF-D detected by VEGFR-2-Ig and VEGFR-3-Ig. The VEGFR-1-Ig provides a negative control because VEGFR-1 does not bind VEGF-D.

These results demonstrate that the mature form of VEGF-D generated by furin binds to the extracellular domains of VEGFR-2 and VEGFR-3, as does the previously characterized mature form generated by 293EBNA cells (Stacker et al, J. Biol. Chem. 274 32 27-32136, 1999).

### Example 6

### Processing of VEGF-D is Critical for Binding to VEGFR-2

In order to monitor the effect of VEGF-D processing on receptor binding, a mutant of full-length VEGF-D, tagged with the FLAG octapeptide, was generated that cannot be processed at all. (See generally_PCT/US2006/036357, filed Sep. 19, 2006 incorporated herein by reference in their entirety.) This mutant was created by mutating arginine residues at positions 85 and 88 (adjacent to the site at which the N-terminal propeptide is most commonly cleaved from the VHD, between positions 88 and 89 (Stacker et al., J. Biol. Chem., 274:32127-32136, 1999) and at positions 204 and 205 (adjacent to the site at which the C-terminal propeptide is cleaved from the VHD between positions 205 and 206 (Stacker et al. 1999, supra) to serine residues (FIG. 10A). The DNA encoding VEGF-D-FULL-N-FLAG was subjected to mutagenesis by PCR with the Quikchange Site-Directed Mutagenesis Kit (Stratagene) essentially as described by the manufacturer. The R88S mutation was introduced by amplification with the following primers: 5′-GCA TCC CAT CGG TCC ACT TCC TTT GCG GCA ACT TTC TAT G-3′ (SEQ ID NO: 9) and 5′-CAT AGA AAG TTG CCG CAA AGG AAG TGG ACC GAT GGG ATG C-3′ (SEQ ID NO: 10); the R85S mutation was made in the resulting plasmid by amplification with 5′-CTC GCT CAG CAT CCC ATT CCT CCA CTT CCT TTG CGG-3′ (SEQ ID NO: 11) and 5′-CCG CAA AGG AAG TGG AGG AAT GGG ATG CTG AGC GAG-3′ (SEQ ID NO: 12) and the R204S and R205S mutations were both introduced into the resulting plasmid by amplification with 5′-CCA TAC TCA ATT ATC AGC AGC TCC ATC CAG ATC CCT GAA G-3′ (SEQ ID NO: 13) and 5′-CTT CAG GGA TCT GGA TGG AGC TGC TGA TAA TTG AGT ATG G-3′ (SEQ ID NO: 14). The desired mutations, as well as the absence of any unwanted mutations, in the resulting DNA fragments were confirmed by nucleotide sequencing. The resulting mutant derivative was designated VEGF-D_{SSTS.IISS} and was expressed in 293EBNA cells in comparison to a full-length form of VEGF-D in which the sequences at the cleavage sites had not been mutated (this derivative was previously designated VEGF-D-FULL-N-FLAG—see FIG. 10A, (Stacker et al. 1999, supra).

As expected, a portion of VEGF-D-FULL-N-FLAG in the conditioned culture media was proteolytically processed as indicated by the presence of ˜31 kDa and ˜21 kDa bands in addition to full-length material of ˜50 kDa (FIG. 10B). The ˜31 kDa band was shown previously to be a species consisting of the N-terminal propeptide and the VHD whereas the ˜21 kDa band corresponds to mature VFGF-D (Stacker et al., supra). In contrast, all of the VEGF-D_{SSTS.IISS} from the conditioned culture media consisted of the full-length ˜50 kDa form (FIG. 10B). This finding demonstrated that the mutations in VEGF-D_{SSTS.IISS} prevent cleavage of both propeptides from the VHD and that proteolytic processing is not required for VEGF-D to be secreted from the cell. In addition, these results show that mutation of the site most commonly used for cleavage of the N-terminal propeptide from the VHD (the “Major” site—see FIG. 10A) is sufficient to completely block cleavage of the N-terminal propeptide, suggesting that in wild-type VEGF-D cleavage at the “Minor” site may only occur after cleavage at the “Major” site.

The VHD of VEGF-D can have an alternative N-terminus to that generated by cleavage of the N-terminal propeptide at the cleavage site analyzed in this Example (Stacker et al. 1999, supra). The alternative N-terminus is 11 amino acids C-terminal from the cleavage site studied here. The majority of mature VEGF-D detected in the conditioned medium of 293EBNA cells secreting full-length VEGF-D is generated by proteolysis at the cleavage site studied in this Example. Therefore, this site is known as the “Major” site and the alternative cleavage site is considered the “Minor” site (Stacker et al. 1999, supra). The sequence of the “Minor” site does not contain the basic residues required for PC-mediated cleavage, indicating that PCs are not responsible for proteolysis at this site. Inhibition of VEGF-D processing with dec-RVKR-CMK completely abrogated proteolytic cleavage of the N-terminal propeptide from the VHD, i.e. at both cleavage sites, suggesting that PC-induced cleavage of the N-terminal propeptide at the “Major” site may be a prerequisite for subsequent cleavage at the “Minor” site by another protease.

To determine the relative affinities of the ligands VEGF-D_{SSTS.IISS}, VEGF-D-FULL-N-FLAG and mature VEGF-D (designated VEGF-DΔNΔC) for VEGFR-2 and VEGFR-3, the relative binding kinetics for these interactions was analyzed by biosensor analysis using surface plasmon resonance detection (Nice et al., Bioessays, 21:339-352, 1999). The preparations of VEGF-D_{SSTS.IISS} and VEGF-DΔNΔC used for this analysis each consisted of one VEGF-D species devoid of other forms of VEGF-D but the preparation of VEGF-D-FULL-N-FLAG did contain traces of partially processed as well as full-length material, reflecting the capacity of wild-type full-length VEGF-D to be proteolytically processed in the 293EBNA cell cultures) from which these proteins were purified (see FIG. 10B). The binding constants for the interactions were obtained by analysis of the initial dissociation phase to obtain the kd, which was then used to constrain a global analysis of the association region of the curves, using regions of the curves where a 1:1 Langmurian model appeared to be operative. Significantly, the apparent affinity of the interaction between mature VEGF-D and VEGFR-2 appeared ˜17,000-fold greater than that for the unprocessed mutant VEGF-D_{SSTS.IISS} (FIG. 10C), indicating the critical importance of processing for the binding of VEGF-D to this receptor. In contrast, the affinity of mature VEGF-D for VEGFR-3 was only ˜18-fold greater than that for VEGF-D_{SSTS.IISS}. The affinity of the VEGF-D-FULL-N-FLAG preparation for VEGFR-2 was ˜94-fold greater than that for VEGF-D_{SSTS.IISS}, reflecting the presence of processed derivatives in the preparations of VEGF-D-FULL-N-FLAG.

The receptor binding studies reported here, employing a mutant form of full-length VEGF-D that cannot be proteolytically processed, demonstrate that processing is extremely important for the interaction of VEGF-D with VEGFR-2, the receptor that signals for angiogenesis. This Example demonstrates that mature VEGF-D has a ˜17,000-fold greater affinity for VEGFR-2 than unprocessed VEGF-D. It was previously reported that mature VEGF-D has a ˜290-fold greater affinity for VEGFR-2 than full-length VEGF-D. However, the analysis was conducted with a form of full-length VEGF-D that could be processed. Hence, the sample used for analysis could have been contaminated with partially processed VEGF-D or mature VEGF-D (Stacker et al., 1999, supra). Thus, the effect of VEGF-D processing on the affinity for VEGFR-2 was likely underestimated in the earlier studies.

The fact that processing has such a dramatic effect on VEGFR-2 binding is consistent with previous studies based on adenoviral delivery into rabbit hind limb skeletal muscle, which showed that full-length VEGF-D could promote lymphangiogenesis but not angiogenesis, whereas mature VEGF-D, lacking both propeptides, promoted both lymphangiogenesis and angiogenesis (Rissanen et al., Cir. Res., 92:1098-1106). Given that VEGFR-2 signals predominantly for angiogenesis (Millauer et al., Cell, 72:835-846, 1993) whereas VEGFR-3 signals predominantly for lymphangiogenesis (Veikkola et al., EMBO J, 20:1223-1231, 2001)., the in vivo results in this Example correlate well with the capacity of mature VEGF-D to bind both VEGFR-2 and VEGFR-3 with high affinity in contrast to full-length VEGF-D which binds only VEGFR-3 with high affinity.

### Example 7

### Role of Furin in VEGF-D Processing

Given that furin is considered to be a very broadly expressed PC and its up-regulation has been correlated with tumor progression and invasiveness (Khatib et al., Am. J. Pathol., 160:1921-1935, 2002), the involvement of furin in VEGF-D processing was studied further by targeting furin mRNA in HeLa cells using shRNA.

Short hairpin RNA (shRNA) primers targeting furin RNA were designed using an in-house primer design algorithm. These primer pairs form the shRNA structure that then results in functional siRNA targeting sequences 457-476 and 558-575 of open reading frame of human furin sequence (Genbank Accession No. NM_{—}002569.2, SEQ ID NO: 21). The underlined sections are structural motifs for the formation of the small hairpin RNA structure and for the ligation of the annealed primers to the parental vector. In bold the 5′-3′ upper strand of the target sequence which is naturally a part of the primer sequence.

1. First primer pair offering an almost complete knockdown of protein expression in vitro:

primer pair targeting bases 457-476 of human furin

(SEQ ID NO:15)

5′-GACGATGGCATCGAGAAGAATTCAAGAGATTCTTCTCGATGCCATCG
TC TTTTTT-3′

(SEQ ID NO:16)

5′-AATTAAAAAA GACGATGGCATCGAGAAGAA TCTCTTGAATTCTTC
TCGATGCCATCGTC-3′

2. Second shRNA pair offering a ca. 70-80% knockdown of protein expression in vitro:

primer pair targeting bases 558-575 of human furin

(SEQ ID NO:17)

5′-CACACAGATGAATGACAA TTCAAGAGA TTGTCATTCATCTGTGTG
TTTTTT-3′

(SEQ ID NO:18)

5′-AATTAAAAAA CACACAGATGAATGACAA TCTCTTGAATTGTCATT
CATCTGTGTG-3′

The above shRNA primers were generated to target the sequences 5′-GAC GAT GGC ATC GAG AAG AA-3′ (SEQ ID NO: 19) and 5′-ACA CAC AGA TGA ATG ACA A-3′ (SEQ ID NO: 20). Primers were annealed by heating a mix of 60 pM of both primers in 50 μl annealing buffer (0.1 M potassium acetate, 2 mM magnesium acetate, 30 mM HEPES pH 7.4) at 94° C. for 4 min then 70° C. for 10 min and allowed to cool to room temperature. Annealing was verified in 3% low melting point agarose gel. The shRNA construct was then ligated into pMSCVpac mU6 which is a derivative of pMSCV (Clontech). The shRNAs were validated by assessing reduction of furin protein levels resulting from co-transfections of shRNA and furin expression constructs in 293EBNA cells or other cell lines stably overexpressing furin.

The furin shRNA construct used was demonstrated to reduce furin protein levels in the cell considerably (FIG. 8C). Furthermore, this shRNA construct almost totally blocked VEGF-D processing in HeLa cell cultures as the relative abundance of mature VEGF-D (˜21 kDa) and of the species consisting of the N-terminal propeptide and the VHD (˜31 kDa) was greatly reduced in comparison to full-length VEGF-D (˜50 kDa) in the presence of the furin shRNA construct (FIG. 8C).

### Example 7

### Localization of VEGF-D and Furin In Vivo

The previous Examples indicate that furin can promote proteolytic processing of VEGF-D when these molecules are expressed in cell lines in culture. This Example demonstrates that VEGF-D and furin are similarly localized in human tissue, such that furin processing of VEGF-D could occur in vivo.

Given the role of VEGF-D as an angiogenic and lymphangiogenesis growth factor, this issue was explored by studying the localization of VEGF-D and furin in the human aorta that contains a well-developed lymphatic network in its adventitia. The lymphatic network at the outer edge of the adventitia was detected using the D2-40 antibody, that binds to the mucin-type glycoprotein podoplanin and stains the endothelium of lymphatic vessels but not of blood vessels (Khatib et al., Am. J. Pathol., 16:1921-1935, 2002; Schacht et al., Am. J. Pathol., 166:913-921 2005). VEGF-D was detected near these lymphatic vessels in a region of what histopathalogically appeared to be a perivascular mononuclear inflammatory cell infiltrate around and adjacent to small vessels in the adventitia. The mononuclear inflammatory cells were also positive for furin. These overlapping staining patterns indicate that, at least in inflammatory cells located in this region of tissue, furin and VEGF-D can be expressed in the same cells so these proteins are in sufficiently close proximity for a direct interaction to occur between them. The expression of these proteins were explored further in human endometrium, a tissue rich in blood vessels, using thin serial sections to allow identification of cells expressing both VEGF-D and furin. Many cells were identified associated with the luminal epithelium and the stroma that were immunopositive for both VEGF-D and furin demonstrating that these two proteins can be located in the same cells in vivo.

Although furin is localized within the trans-Golgi network, it also undergoes regulated trafficking through the endosomes to the cell surface (Thomas et al., Nat. Rev. Mol. Cell Biol., 3:753-766, 2002; Nakayama et al., Biochem. J., 327:625-635, 1997; Molloy et al., Trends Cell Biol., 9:28-35, 1999) and a soluble form of active furin has been reported in the medium of several cell types (Wise et al., Proc. Natl. Acad. Sci. UDS, 87:9378-9382, 1990; Vidricaire et al., Biochem. Biophys. Res. Commun., 195:1011-1018, 1993; Brandeis et al., Nature, 371:435-438, 1994), possibly the product of proteolytic removal of the furin transmembrane region by an undetermined protease. Hence, the localization of furin is consistent with the observation that VEGF-D is processed outside the cell or at the cell surface. Furthermore, purified full-length VEGF-D can be processed to the mature form in cell-free conditioned media in a PC-dependent manner suggesting that a secreted or shed form of furin, or of another PC, contributes to VEGF-D processing.

Conclusions

This study demonstrates that PCs can promote activation of VEGF-D and identifies three members of this family of proteases capable of this process. Furin and PC5 promote cleavage of both propeptides of VEGF-D from the VHD, indicating that these enzymes can fully activate this growth factor. In contrast, PC7 promotes cleavage of the C-terminal propeptide, not the N-terminal propeptide, from the VHD. Hence, PC7 can only partially activate VEGF-D. The findings that PCs can be expressed in cancer (Khatib et al, Am. J. Pathol. 160 1921-1935, 2002), as can VEGF-C and VEGF-D, indicate that this proteolytic activation mechanism, that dramatically enhances the affinities of VEGF-C and VEGF-D for VEGFR-2 and VEGFR-3, may be important for promoting tumor angiogenesis and lymphangiogenesis, thereby facilitating solid tumor growth and metastatic spread. This proteolytic activation of these growth factors is therefore a promising target for novel anti-cancer therapeutics. Furthermore, the findings that PCs also activate other growth factors that can be important for tumor progression, such as TGF-β and PDGF-A (Dubois et al, J. Biol. Chem. 270 10618-10624, 1995; Siegfried et al, Cancer Res. 63 1458-1463, 2003), make the PCs even more attractive therapeutic targets.

Given that furin is considered to be a very broadly expressed PC and its up-regulation has been correlated with tumor progression and invasiveness (for review see (19)), we further explored the involvement of this enzyme in VEGF-D processing by targeting its expression in HeLa cells using shRNA. This demonstrated that knockdown of furin led to a significant reduction in the degree of VEGF-D processing. This was surprising as other enzymes (e.g. other PCs and plasmin) can also process VEGF-D so it was considered unlikely that targeting only furin would be sufficient to significantly block processing. The observation that targeting furin can have a profound effect on VEGF-D processing raises the possibility that targeting this enzyme alone may be an effective approach for restricting the action of VEGF-D in pathological scenarios in vivo, e.g. cancer.

### Example 8

### Effects of Furin or Broad-Range PC Inhibitor on Biological Effects of VEGF-D

In order to test if a furin or broad-range PC inhibitor can block the biological effects of VEGF-D, mammalian cells are employed that proteolytically process VEGF-D, such as 293EBNA or Capan-1 pancreatic carcinoma cells. Derivatives of these cells harboring an expression construct for full-length VEGF-D are transfected with the shRNA construct for furin (as described here) or treated with a broad-range PC inhibitor such as decanoyl-Arg-Val-Lys-Arg-chloromethylketone (see, PCT WO 2005/112971 A1, the disclosure of which is incorporated herein by reference in its entirety). The inhibition of VEGF-D processing by these agents is confirmed by immunoprecipitation/Western blotting of the conditioned cell culture media using antibody that binds the VHD of VEGF-D. The conditioned cell culture media from cells treated with the inhibitors (and from cells in which these agents are omitted, i.e. “mock” treatments) are used for incubations with BaF3 pre-B cells expressing chimeric receptors consisting of the extracellular domains of VEGFR-2 or VEGFR-3 and the transmembrane and cytoplasmic domains of the erythropoietin receptor (EpoR) (Stacker et al., J. Biol. Chem., 274:34884-34892, 1999; Achen et al., Eur. J. Biochem., 267:2505-2515, 2000). These BaF3 cells rely on binding and cross-linking of the chimeric receptors to induce signaling within the cells that promotes survival and proliferation—in the absence of this signaling these cells die. Hence, these cells provide bioassays for binding and cross-linking of the extracellular domains of VEGFR-2 and VEGFR-3. The proliferation of the cells in response to the conditioned media containing VEGF-D is monitored by incorporation of [3H]thymidine into DNA. When the cells expressing VEGF-D are treated with shRNA vector targeting furin mRNA or decanoyl-Arg-Val-Lys-Arg-chloromethylketone or other PC inhibitors, the processing of VEGF-D is blocked and the conditioned media do not induce significant proliferation of the BaF3 cells expressing the VEGFR-2/EpoR or VEGFR-3/EpoR chimeric receptors. In contrast, conditioned media from the “mock” controls contain some VEGF-D that is partially or fully processed, and induce proliferation of the BaF3 cell lines. This demonstrates that furin or broad-range PC inhibitors do prevent VEGF-D from binding and cross-linking its receptors which is an essential step for the biological activities of this growth factor.

### Example 9

### Alternative Approaches for Monitoring the Effect of Furin Inhibitors or Broad-Range PC Inhibitors on the Biological Activity of VEGF-D

Alternative approaches for monitoring the effect of furin inhibitors or broad-range PC inhibitors on the biological activity of VEGF-D are to use the conditioned media described in Example 8 to promote proliferation of endothelial cells—this is monitored using mixed populations of cells such as bovine aortic endothelial cells or human umbilical vein endothelial cells (Achen et al., Proc. Natl. Acad. Sci. USA, 95:548-553, 1998; Breier et al., Development, 114:521-532, 1992) or more pure populations such as lymphatic endothelial cells (Makinen et al., EMBO J., 20:4762-4773, 2001). These endothelial cells are also used with the conditioned media in assays or tube formation and cell migration (Makinen et al., EMBO J., 20:4762-4773, 2001; Montesano et al., J. Cell Biol., 97:1648- 1652, 1983). An alternative to using the conditioned media from the cells producing VEGF-D is to purify the VEGF-D from these media by affinity chromatography (Stacker et al., J. Biol. Chem., 274:32127-32136, 1999) and then to use the VEGF-D at higher concentrations in these assays. In addition, the conditioned media or VEGF-D purified from them are used in assays of angiogenesis or lymphangiogenesis such as those established in the chick chorioallantoic membrane (Oh et al., Dev. Biol., 188:96-109, 1997) or the mouse ear (Nagy et al., J. Exp. Med., 196:1497-1506, 2002).

### Example 10

### Assay of VEGF-D Blockade in Angiogenesis and Lymphangiogenesis

There continues to be a long-felt need for additional agents that inhibit angiogenesis (e.g., to inhibit growth of tumors). Moreover, various angiogenesis inhibitors may work in concert through the same or different receptors, and on different portions of the circulatory system (e.g., arteries or veins or capillaries; vascular or lymphatic). Angiogenesis assays are employed to measure the effects of proprotein convertase and furin inhibitors on angiogenic processes, alone or in combination with other angiogenic and anti-angiogenic factors to determine preferred combination therapy involving proprotein convertase or furin inhibitors. Exemplary procedures include the following.

A. In vitro Assays for Angiogenesis

1. Sprouting Assay

HMVEC cells (passage 5-9) are grown to confluency on collagen coated beads (Pharmacia) for 5-7 days. The beads are plated in a gel matrix containing 5.5 mg/ml fibronectin (Sigma), 2 units/ml thrombin (Sigma), DMEM/2% fetal bovine serum (FBS) and the following test and control proteins: 20 ng/ml VEGF, 20 ng/ml VEGF-C, 20 ng ml VEGF-D, or growth factors plus proprotein convertase or furin inhibitors, and several combinations of other angiogenic and anti-angiogenic factors. Serum free media supplemented with test and control proteins is added to the gel matrix every 2 days and the number of endothelial cell sprouts exceeding bead length are counted and evaluated.

2. Migration Assay

The transwell migration assay previously described may also be used in conjunction with the sprouting assay to determine the effects the proprotein convertase or furin inhibitors of the invention have on the interactions of VEGF-D activators and cellular function. The effects of VEGF-D on cellular migration are assayed in response the proprotein convertase or furin inhibitors, or in combination with known angiogenic or anti-angiogenic agents. A decrease in cellular migration due to the presence of proprotein convertase or furin inhibitors after VEGF-D stimulation indicates that the invention provides a method for inhibiting angiogenesis.

This assay may also be carried out with cells that naturally express either VEGFR-3 or VEGFR-2. Use of naturally occurring or transiently expressing cells displaying a specific receptor may determine that the proprotein convertase or furin inhibitors of the invention may be used to preferentially treat diseases involving aberrant activity of either VEGFR-3 or VEGFR-2.

B. In Vivo Assays for Angiogenesis and Lymphangiogenesis

1. Chornoallantoic Membrane (CAM) Assay

Three-day old fertilized white Leghorn eggs are cracked, and chicken embryos with intact yolks are carefully placed in 20×100 mm plastic Petri dishes. After six days of incubation in 3% CO₂ at 37° C., a disk of methylcellulose containing VEGF-D, and various combinations of the proprotein convertase or furin inhibitors, and soluble VEGFR-2 or VEGFR-3 complexes, dried on a nylon mesh (3×3 mm) is implanted on the CAM of individual embryos, to determine the influence of proprotein convertase or furin inhibitors on vascular development and potential uses thereof to promote or inhibit vascular formation. The nylon mesh disks are made by desiccation of 10 μl of 0.45% methylcellulose (in H₂O). After 4-5 days of incubation, embryos and CAMs are examined for the formation of new blood vessels and lymphatic vessels in the field of the implanted disks by a stereoscope. Disks of methylcellulose containing PBS are used as negative controls. Antibodies that recognize both blood and lymphatic vessel cell surface molecules are used to further characterize the vessels.

2. Corneal Assay

Corneal micropockets are created with a modified von Graefe cataract knife in both eyes of male 5- to 6-week-old C57BL6/J mice. A micropellet (0.35×0.35 mm) of sucrose aluminum sulfate (Bukh Meditec, Copenhagen, Denmark) coated with hydron polymer type NCC (IFN Science, New Brunswick, N.J.) containing various concentrations of VEGF molecules (especially VEGF-D) alone or in combination with: i) factors known to modulate vessel growth (e.g., 160 ng of VEGF, or 80 ng of FGF-2) ; or ii) proprotein convertase or furin inhibitors. The pellet is positioned 0.6-0.8 mm from the limbus. After implantation, erythromycin/ophthamic ointment is applied to the eyes. Eyes are examined by a slit-lamp biomicroscope over a course of 3-12 days. Vessel length and clock-hours of circumferential neovascularization and lymphangiogenesis are measured. Furthermore, eyes are cut into sections and are immunostained for blood vessel and/or lymphatic markers (LYVE-1 [Prevo et al., J. Biol. Chem., 276:19420-19430, 2001)], podoplanin [Breiteneder-Geleff et al., Am. J. Pathol., 154:385-94, 1999).] and VEGFR-3) to further characterize affected vessels.

### Example 11

### Effect of Proprotein Convertase and Furin Inhibitors On VEGF-D Mediated Tumor Growth and Metastasis

To demonstrate the ability of proprotein convertase and furin inhibitors of the invention employed to inhibit tumor growth and/or metastasis, any accepted tumor model may be employed. Exemplary models include animals predisposed to developing various types of cancers, animals injected with tumors or tumor cells or tumor cell lines from the same or different species, including optionally cells transformed to recombinantly overexpress one or more growth factors such as VEGF-A, VEGF-B, VEGF-C, VEGF-D, or VEGF-E, or PDGF-A, or PDGF-B, or PDGF-C, or PDGF-D or P1GF. To provide a model for tumors in vivo in which multiple growth factors are detectable, it is possible to transform tumor cell lines with exogenous DNA to cause expression of multiple growth factors.

The proprotein convertase or furin inhibitors may be administered directly, e.g., in protein form by i.v. transfusion or by implanted micropumps, or in nucleic acid form as part of a gene therapy regimen. Subjects are preferably grouped by sex, weight, age, and medical history to help minimize variations amongst subjects.

Efficacy is measured by a decrease in tumor, size (volume) and weight. One may also examine the nature of the effect on tumor size, spreads (metasteses) and number of tumors. Animals may be looked at as a whole for survival time and changes in weight. Tumors and specimens are examined for evidence of augiogenesis, lymphangiogenesis, and/or necrosis.

SCID mice may be used as subjects for the ability of the proprotein convertase and furin inhibitors of the present invention to inhibit or prevent the growth of tumors. The proprotein convertase or furin inhibitors used in the therapy is generally chosen such that it binds to a growth factor ligand expressed by the tumor cell, especially growth factors that are overexpressed by the tumor cell relative to non-neoplastic cells in the subject. In the SCID model, tumor cells, e.g., MCF-7 cells, may be transfected with a virus encoding a particular growth factor under the control of a promoter or other expression control sequence that provides for overexpression of the growth factor as described in WO 02/060950. Alternatively, other cell lines may be employed, e.g., HT-1080, as described in U.S. Pat. No. 6,375,929. One may transfect the tumor cells with as may growth factor ligands as one desires to overexpress, or a tumor cell line may be chosen that already overexpresses one or more growth factor ligands of interest. One group of subjects is implanted with cells that have been mock-transfected, i.e., with a vector lacking a growth factor ligand insert.

Either before, concurrently with, or after the tumor implantation of the above-described cells, subjects are treated with a proprotein convertase or furin inhibitor. There are a number of different ways of administering the inhibitor. In vivo and/or ex vivo gene therapy may be employed. For example, cells may be transfected with a adenovirus, or other vector, that encodes the inhibitor and implanted with the tumor cells expressing the growth factor(s), the cells transfected with the inhibitor may be the same as those transformed with growth factor(s) (or already overexpressing the growth factor(s)). In some embodiments, an adenovirus that encodes that inhibitor is injected in vivo, e.g., intravenously. In some embodiments, the inhibitor itself (e.g., in protein form) is administered either systematically or locally, e.g., using a micropump. When testing the efficacy of a particular inhibitor, at least one control is normally employed.

Exemplary Procedures

A. Preparation Of Plasmid Expression Vectors, Transfection of Cells, and Testing of the Same

A cDNA encoding VEGF-A, VEGF-B, VEGF-C, VEGF-D, P1GF, PDGF-A, PDGF-B, PDGF-C, PDGF-D, or combinations thereof introduced into a pEBS7 plasmid (Peterson and Legerski, Gene, 107: 279-84, 1991.). This same vector may be used for the expression of the proprotein convertase or furin inhibitors.

The MCF-7S1 subclone of the human MCF-7 breast carcinoma cell line is transfected with the plasmid DNA by electroporation and stable cell pools are selected and cultured as previously described (Egeblad and Jaattela, Int. J. Cancer, 86: 617-25, 2000). The cells are metabolically labeled in methionine and cysteine free MEM (Gibco) supplemented with 100 μCi/ml [35S]-methionine and [35S]-cysteine (Redivue Pro-Mix, Amersham Pharmacia Biotech). The labeled growth factors are immunoprecipitated from the conditioned medium using antibodies against the expressed growth factor(s). The immunocomplexes and the binding complexes are precipitated using protein A sepharose (Amersham Pharmacia Biotech), washed twice in 0.5% BSA, 0.02% Tween 20 in PBS and once in PBS and analyzed in SDS-PAGE under reducing conditions.

B. Subject Preparation and Treatment

Cells (20,000/well) are plated in quadruplicate in 24-wells, trypsinized on replicate plates after 1, 4, 6, or 8 days and counted using a hemocytometer. Fresh medium is provided after 4 and 6 days. For the tumorgenesis assay, sub-confluent cultures are harvested by trypsination, washed twice and 107 cells in PBS are inoculated into the fat pads of the second (axillar) mammary gland of ovariectomized SCID mice, carrying subcutaneous 60-day slow-release pellets containing 0.72 mg 17β-estradiol (Innovative Research of America). The ovarectomy and implantation of the pellets are performed 4-8 days before tumor cell inoculation.

The cDNA coding for the proprotein convertase or furin inhibitor is subcloned into the pAdBgIII plasmid and the adenoviruses produced as previously described (Laitinen et al., Hum. Gene Ther., 9: 1481-6, 1998). The proprotein convertase or furin inhibitor or LacZ control (Laitinen et al., Hum. Gene Ther., 9: 1481-6, 1998) adenoviruses, 109 pfu/mouse, are injected intravenously into the SCID mice 3 hours before the tumor cell inoculation.

C. Analysis of Treatment Efficacy

Tumor length and width are measured twice weekly in a blinded manner, and the tumor volume are calculated as the length×width×depth×0.5, assuming that the tumor is a hemi-ellipsoid and the depth is the same as the width (Benz et al., Breast Cancer Res. Treat., 24: 85-95, 1993).

The tumors are excised, fixed in 4% paraformaldehyde (pH 7.0) for 24 hours, and embedded in paraffin. Sections (7 μm) are immunostained with monoclonal antibodies against, for example, PECAM-1 (Pharmingen), VEGFR-1, VEGFR-2, VEGFR-3 (Kubo et al., Blood, 96: 546-553, 2000) or PCNA (Zymed Laboratories), PDGFR-α, PDGFR-β or polyclonal antibodies against LYVE-1 (Banerji et al., J Cell Biol, 144: 789-801, 1999), VEGF-C (Joukov et al., EMBO J., 16: 3898-911, 1997), laminin according to published protocols (Partanen et al., Cancer, 86: 2406-12, 1999), or any of the growth factors. The average of the number of the PECAM- 1 positive vessels are determined from three areas (60×magnification) of the highest vascular density (vascular hot spots) in a section. All histological analyses are performed using blinded tumor samples.

Three weeks after injection of adenoviris constructs and/or protein therapy, four mice from each group are narcotized, the ventral skin is opened and a few microliters 3% Evan's blue dye (Sigma) in PBS is injected into the tumor. The drainage of the dye from the tumor is followed macroscopically.

Imaging and monitoring of blood and blood proteins to provide indication of the health of subjects and the extent of tumor vasculature may also be performed.

### Example 12

### Effects On Tumor Progression In Subjects Using a Combined Therapy of a Proprotein Convertase or Furin Inhibitor and a Chemotherapeutic Agent

This study is carried out to test the efficacy of using the proprotein convertase and furin inhibitors of the invention in combination with other anti-cancer therapies. Such therapies include chemotherapy, radiation therapy, anti-sense therapy, RNA interference, and monoclonal antibodies directed to cancer targets. The combinatorial effect may be additive, but it is preferably synergistic in its anti-cancer effects, e.g., prevention, suppression, regression, and elimination of cancers, prolongation of lift, and/or reduction in side-effects.

Subjects are divided into groups with one group receiving a chemotherapeutic agent, one group receiving a proprotein convertase or furin inhibitor, and one group receiving both a chemotherapeutic agent and a proprotein convertase or furin inhibitor at regular periodic intervals, e.g., daily, weekly or monthly. In human studies, the subjects are generally grouped by sex, weight, age, and medical history to help minimize variations among subjects. Ideally, the subjects have been diagnosed with the same type of cancer. In human or non-human subjects, progress can be followed by measuring tumor size, metastases, weight gain/loss, vascularization in tumors, and white blood cells counts.

Biopsies of tumors are taken at regular intervals both before and after beginning treatment. For example, biopsies are taken just prior to treatment, at one week, and then at one month intervals, thereafter, or whenever possible, e.g., as tumors are excised. One examines the biopsies for cell markers, and overall cell and tissue morphology to assess the effectiveness of the treatment. In addition, or in the alternative, imaging techniques may be employed.

For non-human animal studies, an additional placebo control may be employed. Animal studies, performed in accordance with NIH guidelines, also provide the advantage of the insertion of relatively uniform cancer cell population, and tumors that selectively overproduce the one or more growth factors targeted by the proprotein convertase or furin inhibitor.

For the sake of completeness of disclosure, all literature articles cited herein are expressly incorporated in this specification by reference.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of inhibiting angiogenesis or lymphangiogenesis comprising administering to an organism in need of inhibition of angiogenesis or lymphangiogenesis a composition that comprises a proprotein convertase inhibitor effective to inhibit angiogenesis or lymphangiogenesis.

2. The method of claim 1, wherein the proprotein convertase inhibitor is an inhibitor specific for furin.

3. The method of claim 2, wherein the inhibitor comprises a member selected from the group consisting of:
(a) antibody substances that bind to a proprotein convertase polypeptide and that inhibit proprotein convertase function; and
(b) inhibitory nucleic acids.

4. The method of claim 3, wherein the inhibitor comprises an antibody substance selected from the group consisting of: (a) a monoclonal antibody; (b) antigen binding fragments of proprotein convertase antibodies; and (c) domain antibodies (dAbs).

5. The method of claim 3, wherein the inhibitor comprises an antibody fragment selected from the group consisting of Fab fragments, F(ab)2 fragments, Fab′ fragments, F(ab′)2 fragments, Fd fragments, Fd′ fragments, Fv fragments, and single chain antibodies.

6. The method of claim 3, wherein the inhibitor comprises a human or humanized antibody substance.

7. The method of claim 3, wherein the inhibitor is an inhibitory nucleic acid selected from the group consisting of: (a) an antisense oligonucleotide; (b) an inhibitory RNA; (c) a DNA enzyme; (d) a ribozyme; (e) an aptamer.

8. The method of claim 7, wherein the inhibitor is an inhibitory RNA.

9. She method of claim 7, wherein the inhibitory RNA is a short interfering RNA, a double stranded RNA (dsRNA).

10. The method of claim 7, wherein the inhibitory RNA is a short hairpin RNA (shRNA).

11. The method of claim 10, wherein the shRNA comprises at least one sequence selected from the group consisting of SEQ ID Nos: 15, 16, 17 and 18.

12. The method of claim 11, wherein the inhibitor is an inhibitory RNA that targets a portion of the furin cDNA sequence set forth in SEQ ID NO: 19 or SEQ ID NO: 20.

13. The method of claim 3, wherein the composition comprises an expression vector that contains an insert that comprises a nucleotide sequence that encodes the inhibitor.

14. The method of claim 2, wherein the composition further comprises a pharmaceutically acceptable carrier.

15. A method according to claim 2, wherein said organism is a mammal.

16. A method according to claim 2, wherein said organism is human.

17. A method according to claim 2, wherein said organism has a tumor.

18. A method according to claim 2, wherein said organism has macular degeneration.

19. A method for inhibiting angiogenesis or lymphangiogenesis in an organism, comprising:
contacting the organism with a double stranded siRNA molecule under conditions suitable to modulate the expression of a proprotein convertase gene in the organism via RNA interference, wherein a first strand of the double stranded siNA molecule comprises nucleotide sequence having sufficient complementarity to proprotein convertase mRNA, and wherein a second strand of the double stranded siRNA molecule comprises nucleotide sequence having sufficient complementarity to the first strand, for the siRNA molecule to inhibit expression of the proprotein convertase gene via RNA interference.
